SEQUENCE LISTING (13 sequences)

SEQ ID NO: 1 - protein, 16 aa, Artificial Sequence
  Completely synthetic amino acid
RSSQSIVHSNGNTYLQ

SEQ ID NO: 2 - protein, 7 aa, Artificial Sequence
  Completely synthetic amino acid
KVSNRLY

SEQ ID NO: 3 - protein, 9 aa, Artificial Sequence
  Completely synthetic amino acid
FQGSHVPWT

SEQ ID NO: 4 - protein, 6 aa, Artificial Sequence
  Completely synthetic amino acid
GGYLWN

SEQ ID NO: 5 - protein, 16 aa, Artificial Sequence
  Completely synthetic amino acid
YISYDGTNNYKPSLKD

SEQ ID NO: 6 - protein, 8 aa, Artificial Sequence
  Completely synthetic amino acid
YGRVFFDY

SEQ ID NO: 7 - protein, 112 aa, Artificial Sequence
  Completely synthetic amino acid
DVVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIK

SEQ ID NO: 8 - protein, 112 aa, Artificial Sequence [sequence omitted]

SEQ ID NO: 9 - protein, 117 aa, Artificial Sequence
  Completely synthetic amino acid
QVQLQESGPGLVKPSETLSLTCTVSGYSITGGYLWNWIRQPPGKGLEWMGYISYDGTNNYKPSLKDRITISRDTSKNQFSLKLSSVTAADTAVYYCARYGRVFFDYWGQGTLVTVSS

SEQ ID NO: 10 - protein, 117 aa, Artificial Sequence [sequence omitted]

SEQ ID NO: 11 - protein, 117 aa, Artificial Sequence
  Completely synthetic amino acid
QVQLQESGPGLVKPSETLSLTCTVSGYSISGGYLWNWIRQPPGKGLEWIGYISYDGTNNYKPSLKDRVTISVDTSKNQFSLKLSSVTAADTAVYYCARYGRVFFDYWGQGTLVTVSS

SEQ ID NO: 12 - protein, 112 aa, Artificial Sequence
  Completely synthetic amino acid
DVLMTQIPLSLPVSLGDQASISCRSSQSIVHSNGNTYLQWYLQKPGQSPKLLIYKVSNRLYGVPDRFSGSGSGTDFTLKISSVEAEDLGVYYCFQGSHVPWTFGGGTKLEIK

SEQ ID NO: 13 - protein, 117 aa, Artificial Sequence
  Completely synthetic amino acid
DVQLQESGPGLVKPSQSLSLTCSVTGYSITGGYLWNWIRQFPGNKLEWMGYISYDGTNNYKPSLKDRISITRDTSKNQFFLKLNSVTNEDTATYYCARYGRVFFDYWGQGTTLTVSS

(19) United States
(12) Patent Application Publication
(10) Pub. No.: US 2012/0058112 A1
(43) Pub. Date: Mar. 8, 2012

(54) ANTIBODIES

(76) Inventors: Sriram Sathyanarayanan, Natick, MA (US); Shailaja Kashibhatla, San Diego, CA (US); Christopher Winter, Swampscott, MA (US); Michael Chastain, Seattle, WA (US)

(21) Appl. No.: 13/264,595

(22) PCT Filed: Apr. 6, 2010
(86) PCT No.: PCT/US10/30022

§ 371 Date: Oct. 14, 2011

Related U.S. Application Data
(60) Provisional application No. 61/169,768, filed on Apr. 16, 2009.

(51) Int. Cl. A61K 39/395; A61K 31/517; A61K 31/519

(52) U.S. Cl. 424/133.1; 424/145.1; 424/158.1; 514/210.21; 514/265.1; 514/266.4

(57) ABSTRACT

The combination of an IGF-1R antagonist such as a humanized antibody and an anti-proliferative drug is described. In a preferred embodiment, the present invention describes the combination of an IGF-1R antibody and an anti-proliferative drug belonging to the EGFR-inhibitor class, which is preferably erlotinib. The combination according to the present invention is useful for the treatment of tumours, including IGF-1R and/or EGFR mediated or dependent tumours.

### FIELD OF THE INVENTION

The present invention relates to methods and compositions for enhancing anti-tumor activity in a mammal. More particularly, the invention is concerned with combinations comprising an antibody that specifically binds to human IGF-1R and a receptor tyrosine kinase inhibitor. In particular, the invention relates to combination therapy for treating non-small cell lung cancer and other cancers, e.g., pancreatic cancer via administration of an IGF-1R antibody and a tyrosine kinase inhibitor, particularly erlotinib. The methods and the pharmaceutical compositions comprising said combinations or agents can result in superior tumor cell proliferation inhibition than that observed relative to the use of each individual therapeutic agent, yielding more effective treatment than found by administering an individual component alone. A particular aspect provides for the treatment of erlotinib resistant lung cancer.

### BACKGROUND OF THE INVENTION

Lung carcinomas are responsible for the majority of deaths from cancer among men and are overtaking breast carcinomas as the most frequent cause of cancer death among women. The current prognosis for patients with lung cancer is poor. The mortality rate attendant lung cancer deaths have increased ten-fold in both men and women since 1930, primarily due to an increase in cigarette smoking, but also due to an increased exposure to arsenic, asbestos, chromates, chloromethyl ethers, nickel, polycyclic aromatic hydrocarbons and other agents. See Scott, Lung Cancer: A Guide to Diagnosis and Treatment, Addicus Books (2000) and Alberg et al., in Kane et al. (eds.) Biology of Lung Cancer, pp. 11-52, Marcel Dekker, Inc. (1998). The American Cancer Society estimates there will be over 173,550 new cases of lung cancer in 2004. Additionally, there will be an estimated 160,440 deaths from lung cancer in 2004. ACS Website: cancer with the extension org of the world wide web.

Lung cancer may result from a primary tumor originating in the lung or a secondary tumor which has spread from another organ such as the bowel or breast. Although there are over a dozen types of lung cancer, over 90% fall into two categories: small cell lung cancer (SCLC) and non-small cell lung cancer (NSCLC). See Scott, supra. 70-80% are diagnosed as NSCLC. The term “NSCLC” includes the following cell types: epidermoid carcinoma cells, adenocarcinoma cells, and large undifferentiated carcinoma cells. A diagnosis of lung cancer is usually confirmed by biopsy of the tissue.

Treatment approaches and natural history differ for these two diseases. The majority (80%) of cases of lung cancer in the United States are NSCLC. Although advances in the understanding of important clinical and prognostic factors for both NSCLC and SCLC have been made in the past 20 years, there have been minimal improvements in therapeutic results.

NSCLS is generally divided into three types: squamous cell carcinoma, adenocarcinoma and large cell carcinoma. Both squamous cell cancer and adenocarcinoma develop from the cells that line the airways; however, adenocarcinoma develops from the goblet cells that produce mucus. Large cell lung cancer has been thus named because the cells look large and rounded when viewed microscopically, and generally are considered relatively undifferentiated. See Yesner, Atlas of Lung Cancer, Lippincott-Raven (1998). Non-small cell cancer may be divided into four stages. Stage I is highly localized cancer with no cancer in the lymph nodes. Stage II cancer has spread to the lymph nodes at the top of the affected lung. Stage in cancer has spread near to where the cancer started. This can be to the chest wall, the covering of the lung (pleura), the middle of the chest (mediastinum) or other lymph nodes. Stage IV cancer has spread to another part of the body. Stage I-III cancer is usually treated with surgery, with or without chemotherapy. Stage IV cancer is usually treated with chemotherapy and/or palliative care.

A number of chromosomal and genetic abnormalities have been observed in lung cancer. In NSCLC, chromosomal aberrations have been described on 3p, 9p, 11p, 15p and 17p, and chromosomal deletions have been seen on chromosomes 7, 11, 13 and 19. See Skarin (ed.), Multimodality Treatment of Lung Cancer, Marcel Dekker, Inc. (2000); Gemmill et al., pp. 465-502, in Kane, supra; Bailey-Wilson et al., pp. 53-98, in Kane, supra. Chromosomal abnormalities have been described on 1p, 3p, 5q, 6q, 8q, 13q and 17p in SCLC. In addition, the loss of the short arm of chromosome 3p has also been seen in greater than 90% of SCLC tumors and approximately 50% of NSCLC tumors.

A number of oncogenes and tumor suppressor genes have been implicated in lung cancer. See Mabry, pp. 391-412, in Kane, supra and Sclafani et al., pp. 295-316, in Kane, supra. In both SCLC and NSCLC, the p53 tumor suppressor gene is mutated in over 50% of lung cancers. See Yesner, supra. Another tumor suppressor gene, FHIT, which is found on chromosome 3p, is mutated by tobacco smoke. Id.; Skarin, supra. In addition, more than 95% of SCLCs and approximately 20-60% of NSCLCs have an absent or abnormal retinoblastoma (Rb) protein, another tumor suppressor gene. The ras oncogene (particularly K-ras) is mutated in 20-30% of NSCLC specimens and the c-erbB2 oncogene is expressed in 18% of stage 2 NSCLC and 60% of stage 4 NSCLC specimens. See Van Houtte, supra. Other tumor suppressor genes that are found in a region of chromosome 9, specifically in the region of 9p21, are deleted in many cancer cells, including p16.sup.INK4A and p15.sup.INK4B. See Bailey-Wilson, supra; Sclafani et al., supra. These tumor suppressor genes may also be implicated in lung cancer pathogenesis.

In addition, many lung cancer cells produce growth factors that may act in an autocrine or paracrine fashion on lung cancer cells. See Siegfried et al., pp. 317-336, in Kane, supra, Moody, pp. 337-370, in Kane, supra and Heasley et al., 371-390, in Kane, supra. Many NSCLC tumors express epidermal growth factor (EGF) receptors, allowing NSCLC cells to proliferate in response to EGF. Insulin-like growth factor (IGF-1) is elevated in greater than 80% of NSCLC tumors; it is thought to function as an autocrine growth factor.

Although the majority of lung cancer cases are attributable to cigarette smoking, most smokers do not develop lung cancer. Epidemiological evidence has suggested that susceptibility to lung cancer may be inherited in a Mendelian fashion, and thus have an inherited genetic component. Bailey-Wilson, supra. Thus, it is thought that certain allelic variants at some genetic loci may affect susceptibility to lung cancer.

Current therapies for lung cancer are quite limited. Generally, patient options comprise surgery, radiation therapy, and chemotherapy.

Most cases of lung carcinomas are incurable by chemotherapy and radiation therapy. Depending on the type and stage of a lung cancer, surgery may be used to remove the tumor along with some surrounding lung tissue. A lobectomy refers to a lobe (section) of the lung being removed. If the entire lung is removed, the surgery is called a pneumonectomy. Removing only part of a lobe is known as a segmentectomy or wedge resection.

Indeed, the only curative option for patients with NSCLC is local therapy (surgical excision or local irradiation) in patients with early stage disease (I & II) when the tumor is still localized. At diagnosis however, the majority of patients with NSCLC present with advanced disease, which is not curable by surgery alone. In advanced stages of disease, systemic chemotherapy and/or irradiation can produce objective responses and palliation of symptoms, however, they offer only modest improvements in survival. The median survival of patients with non-resectable disease is 6-12 months. Two-year survival rates for stages IIIB and IV NSCLC are 10.8 and 5.4 percent respectively. Likewise, five-year survival rates are 3.9 and 1.3 percent.

If the cancer has spread to the brain, benefit may be gained from removal of the brain metastasis. This involves a craniotomy (surgery through a hole in the skull).

For radiation therapy several methods exist. External beam radiation therapy uses radiation delivered from outside the body that is focused on the cancer. This type of radiation therapy is most often used to treat a primary lung cancer or its metastases to other organs.

Additionally, radiation therapy can be used as a post surgical treatment to kill very small deposits of cancer that cannot be seen or removed during surgery. Radiation therapy can also be used to palliate (relieve) symptoms of lung cancer such as pain, bleeding, difficulty swallowing, and problems caused by brain metastases.

For chemotherapy, cisplatin or a related drug, carboplatin, are the chemotherapy agents most often used in treating NSCLC. Other new chemical entities available for the treatment of NSCLC including paclitaxel (Taxol), docetaxel (Taxotere), topotecan, irinotecan, vinorelbine, and gemcitabine. While these drugs are improvements over prior chemotherapeutic agents (etoposide, cisplatin and carboplatin), the overall cure rate remains low.

The epidermal growth factor receptor (EGFR) is a member of a family of closely related growth factor receptor tyrosine kinases that includes EGFR (ErbB1), HER2/neu (ErbB2), HER3 (ErbB3), and HERO (ErbB4). Upon ligand binding, these receptors homodimerize or heterodimerize leading to autophosphorylation and subsequent activation of intracellular signaling cascades such as the phosphoinositide 3-kinase (PI3K)/Akt, MAPK/Erk, and Jak/Stat signaling pathways, which play major roles in cell proliferation, survival, and transformation and in therapeutic resistance

Downstream of the EGFR, PI3K pathway plays a critical role in regulating cell survival & proliferation. The ErbB3 receptor plays a unique role in activating PI3K pathway. ErbB3 has weak or no tyrosine kinase activity, however, upon heterodimerization with EGFR, it is phosphorylated on tyrosine residues. Tyrosine-phosphorylated ErbB3 directly binds to and activates PI3K. Studies have shown that PI3K/Akt signaling is tightly regulated by EGFR in TKI-sensitive NSCLCs, and EGFR TKIs down-regulate the PI3K/Akt pathway exclusively in those NSCLC cell lines in which they also inhibit growth (Engelman, J. A. et al. Proc. Natl. Acad. Sci. USA 102, 3788-3793 (2005).

Because EGFR is expressed in a majority of non-small cell lung carcinomas (NSCLC), it has been an attractive target for the development of therapeutic agents. The small-molecule EGFR tyrosine kinase inhibitors (TKI), including gefitinib and erlotinib, have been evaluated in clinical trials for patients with NSCLC. Both agents produce partial responses in 10% to 20% of all NSCLC patients. Lung cancers with EGFR mutation and/or amplification are the most likely to shrink in response to EGFR inhibitors. Activating somatic mutations in the EGFR gene have been identified in NSCLC patents. These ‘gain-of-function’ mutations are either substitutions or short, in-frame deletions or insertions clustered around the region encoding the ATP-binding pocket of the receptor's tyrosine kinase domain. In such cancers, EGFR is the major activator of critical growth and survival signaling pathways, and thus these cancers are addicted to EGFR activity. When exposed to EGFR inhibitors, these key growth and survival signaling pathways are aborted, resulting in apoptosis and/or cell cycle arrest.

Recent data suggests new therapeutic approaches targeting signaling pathways involved in cell proliferation, apoptosis, angiogenesis, and metastasis are being investigated. Among the many potential target pathways, the epidermal growth factor (EGF) receptor (EGFR) signaling pathway has been studied most extensively because EGFR overexpression has been observed in a number of solid tumors, including 40% to 80% of non-small cell lung cancers (NSCLC). As noted, supra, researchers have been testing agents that interfere with the epidermal growth factor receptor (EGFR). EGFR, partly because it is expressed at abnormally high levels on the surface of many types of cancer cells, including non-small cell lung cancer. Examples of these experimental EGFR inhibitors are gefitinib (Iressa®), cetuximab (Erbitux®), and erlotinib (Tarceva®).

Resistance to erlotinib or EGFR inhibition therapy has been observed in the clinic due to activating mutations in KRAS gene (Pao, W. et al. PLoS Med. 2, e73 (2005), a critical down stream signaling component in the MAPK signaling pathway. Acquired resistance to erlotinib therapy has been associated in the clinic with secondary mutations in EGFR exon 20 (T790M). Recent studies have also identified cMET an RTK, which phosphorylates ERB3 and confer resistance to erlotinib therapy.

However, the overall response rate to EGFR TKIs is limited, and the mechanisms mediating resistance to the drugs are poorly understood. The small-molecule EGFR tyrosine kinase inhibitors (TKI), including gefitinib and erlotinib, have been evaluated in clinical trials for patients with NSCLC. Both agents produce partial responses in 10% to 20% of all NSCLC patients. In 2004, researchers with a phase II trial involving previously treated NSCLC patients reported that tumors in 12 percent of the participants responded to treatment with erlotinib. It was unclear, however, whether erlotinib helped the patients live any longer.

In 2004, several phase III clinical trials involving patients with non-small cell lung cancer (NSCLC) reported that patients receiving standard chemotherapy plus an EGFR inhibitor (gefitinib or erlotinib) did no better than patients receiving chemotherapy alone. However, that same year researchers with a phase III Canadian trial reported that erlotinib helped NSCLC patients whose cancer was no longer responding to chemotherapy to live about two months longer than those taking a placebo

Erlotinib did not help patients live any longer overall. The median survival for patients taking erlotinib was 10.6 months compared to 10.5 months for the placebo group. Both groups of patients also experienced about the same “time to progression” (the time it took for their cancer to get worse): 5.1 months for the erlotinib group, 4.9 months for the placebo group. Although the anti-cancer compounds described above make a significant contribution to the art there is a continuing search in this field of art for improved anti-cancer pharmaceuticals.

Investigators have hypothesized erlotinib induces EGFR/IGF-IR heterodimerization on the cell membrane, transmitting a survival signal through IGF-IR and its downstream mediators PI3K/Akt and p44/42 MAPK to stimulate mammalian target of rapamycin (mTOR)-mediated synthesis of EGFR and antiapoptotic survivin proteins. Consequently, inactivation of IGF-IR, suppression of mTOR-mediated protein synthesis, or knockdown of survivin protein renders EGFR-overexpressing NSCLC cells sensitive to the erlotinib treatment. See Floriana Morgillo, et al., Cancer Research 66, 10100-10111, Oct. 15, 2006.

However, resistance to erlotinib or EGFR inhibition therapy has been observed in the clinic due to activating mutations in Kras gene (Pao, W. et al. PLoS Med. 2, e73 (2005), a critical down stream signaling component in the MAPK signaling pathway. Acquired resistance to erlotinib therapy has also been associated in the clinic with secondary mutations in EGFR exon 20 (T790M). Recent studies have also identified cMET an RTK, which phosphorylates ERB3 and confer resistance to erlotinib therapy.

Activation of the IGF1R signaling pathway has recently been associated with mediating resistance to Gefitinib, an EGFR TKI (Guix M et al., J Clin Invest. 118 (7): 2609-2619 (2008). The authors isolated gefitinib-resistant (GR) human squamous carcinoma A431 cells by prolonged incubation of A431 cells with an increasing amount of the inhibitor. In the GR cells, the inhibitor reduced the phosphorylation levels of EGFR, ErbB3, and Erk, but not those of Akt. This adaptive change was accompanied by activation of the signaling events mediated by the IGF-1 receptor (IGF-IR), such as phosphorylation of IRS-1 and the interaction of IRS-1 with P13K. The authors went on to show that inhibition of IGF-IR disrupted the association of IRS-1 with PI3K and restored the ability of gefitinib to reduce Akt phosphorylation and to inhibit cell growth (FIG. 1A). Others have hypothesized that multiple receptor tyrosine kinase activation in a cell could contribute to drug resistance to Tarceva. In fact activation of EGFR and cMET has been observed in clinical samples from Tarceva resistant patients. See also Biochemical and Biophysical Research Communications, 355 (3): 700-706 (April 2007.

Insulin-like growth factors (IGF), e.g., insulin-like growth factor-I and -II have been implicated in exerting mitogenic activity on various cell types such as tumor cells. IGFs are structurally similar to insulin, and have been implicated as a therapeutic tool in a variety of diseases and injuries. Insulin-like growth factor-I (IGF-I) is a 7649-dalton polypeptide with a pI of 8.4 that circulates in plasma in high concentrations and is detectable in most tissues(Rinderknecht and Humbel, Proc. Natl. Acad. Sci. USA, 73: 2365 (1976); Rinderknecht and Humbel, J. Biol. Chem., 253: 2769 (1978)). IGF-I stimulates cell differentiation and cell proliferation, and is required by most mammalian cell types for sustained proliferation. These cell types include, among others, human diploid fibroblasts, epithelial cells, smooth muscle cells, T lymphocytes, neural cells, myeloid cells, chondrocytes, osteoblasts and bone marrow stem cells. Each of these growth factors exerts its mitogenic effects by binding to a common receptor named the insulin-like growth factor receptor-1 (IGF1R) (Sepp-Lorenzino, (1998) Breast Cancer Research and Treatment 47:235). See also Klapper, et al., (1983) Endocrinol. 112:2215 and Rinderknecht, et al., (1978) Febs. Lett. 89:283. There is a large body of literature on the actions and activities of IGFs (IGF-1, IGF-2, and IGF variants). See Van Wyk et al., Recent Prog. Horm. Res., 30: 259 (1974); Binoux, Ann. Endocrinol., 41: 157 (1980); Clemmons and Van Wyk, Handbook Exp. PharmacoL, 57: 161 (1981); Baxter, Adv. Clin. Chem., 25:49 (1986); U.S. Pat. No. 4,988,675; WO 91/03253; WO 93/23071).

The IGF system is composed of membrane-bound receptors for IGF-1, IGF-2, and insulin. The Type 1 IGF receptor (IGF-1R) is closely related to the insulin receptor (IR) in structure and shares some of its signaling pathways (Jones and Clemmons, Endocr. Rev., 16: 3-34 (1995); Ullrich et al., Cell 61: 203 212, 1990), and is structurally similar to the insulin receptor (Ullrich et al., EMBO J. 5: 2503 2512, 1986)). The IGF-I receptor is composed of two types of subunits: an alpha subunit (a 130 135 kD protein that is entirely extracellular and functions in ligand binding) and a beta subunit (a 95-kD transmembrane protein, with transmembrane and cytoplasmic domains). The IGF-IR is initially synthesized as a single chain proreceptor polypeptide which is processed by glycosylation, proteolytic cleavage, and covalent bonding to assemble into a mature 460-kD heterotetramer comprising two alpha-subunits and two beta-subunits. The beta subunit(s) possesses ligand-activated tyrosine kinase activity. This activity is implicated in the signaling pathways mediating ligand action which involve autophosphorylation of the beta-subunit and phosphorylation of IGF-IR substrates.

IGF-IR binds IGF I and IGF II with nanomolar affinity, e.g., Kd of 1×10−9 nM but is capable of binding to insulin with an affinity 100 to 1000 times less. Representative nanomolar affinity values may be found in FEBS Letters, vol. 565, pages 19-22 (2004), the entire content of which is incorporated by reference herein.

There is considerable evidence for a role for IGF-I and/or IGF-IR in the maintenance of tumor cells in vitro and in vivo. For example, individuals with “high normal” levels of IGF-I have an increased risk of common cancers compared to individuals with IGF-I levels in the “low normal” range (Rosen et al., Trends Endocrinol. Metab. 10: 136 41, 1999). For a review of the role IGF-I/IGF-I receptor interaction plays in the growth of a variety of human tumors, see Macaulay, Br. J. Cancer, 65: 311 320, 1992. In addition to playing a key role in normal cell growth and development, IGF-1R signaling has also been implicated as playing a critical role in growth of tumor cells, cell transformation, and tumorigenesis. See Baserga, Cancer Res., 55:249-252 (1995); for a review, see Khandwala et al., Endocr. Rev. 21: 215-244 (2000)); Daughaday and Rotwein, Endocrine Rev., 10:68-91 (1989). Recent data impel the conclusion that IGF-IR is expressed in a great variety of tumors and of tumor lines and the IGFs amplify the tumor growth via their attachment to IGF-IR. Indeed, the crucial discovery which has clearly demonstrated the major role played by IGF-IR in the transformation has been the demonstration that the R-cells, in which the gene coding for IGF-IR has been inactivated, are totally refractory to transformation by different agents which are usually capable of transforming the cells, such as the E5 protein of bovine papilloma virus, an overexpression of EGFR or of PDGFR, the T antigen of SV 40, activated ras or the combination of these two last factors (Sell C. et al., Proc. Natl. Acad. Sci., USA, 90: 11217-11221, 1993; Sell C. et al., Mol. Cell. Biol., 14:3604-3612, 1994; Morrione A. J., Virol., 69:5300-5303, 1995; Coppola D. et al., Mol. Cell. Biol., 14:4588-4595, 1994; DeAngelis T et al., J. Cell. Physiol., 164:214-221, 1995). Other key examples supporting this hypothesis include loss of metastatic phenotype of murine carcinoma cells by treatment with antisense RNA to the IGF-1R (Long et al., Cancer Res., 55:1006-1009 (1995)) and the in vitro inhibition of human melanoma cell motility (Stracke et al., J. Biol. Chem., 264:21554-21559 (1989)) and of human breast cancer cell growth by the addition of IGF-1R antibodies (Rohlik et al., Biochem. Biophys. Res. Commun., 149:276-281 (1987)).

Other arguments in favor of the role of IGF-IR in carcinogenesis come from studies using murine monoclonal antibodies directed against the receptor or using negative dominants of IGF-IR. In effect, murine monoclonal antibodies directed against IGF-I R inhibit the proliferation of numerous cell lines in culture and the growth of tumor cells in vivo (Arteaga C. et al., Cancer Res., 49:6237-6241, 1989; Li et al., Biochem. Biophys. Res. Com., 196:92-98, 1993; Zia F et al., J. Cell. Biol., 24:269-275, 1996; Scotlandi K et al., Cancer Res., 58:4127-4131, 1998). It has likewise been shown in the works of Jiang et al. (Oncogene, 18:6071-6077, 1999) that a negative dominant of IGF-IR is capable of inhibiting tumor proliferation.

IGF-IR levels are elevated in tumors of lung (Kaiser et al., J. Cancer Res. Clin. Oncol. 119: 665 668, 1993; Moody et al., Life Sciences 52: 1161 1173, 1993; Macauley et al., Cancer Res., 50: 2511 2517, 1990).

As indicated above, many therapeutics are recommended for use in combination as a first-line therapy or only if other therapeutics have failed as second-, and third-line agents. While there are many compounds in ongoing or recently completed therapeutic trials, there is great need for additional therapeutic compounds capable of treating early stage and advanced or metastasized lung cancer.

The 5-year survival rate for lung cancer patients remains extremely poor (515), underscoring the need for more effective treatment strategies. Previous attempts to develop an effective therapy for treating NSCLC, especially Erlotinib resistant cancers have not been reported. Described herein are novel combination therapeutics or combination regiments that meet this need.

Other features and advantages of the invention will be apparent from the detailed description and examples that follow.

### SUMMARY OF THE INVENTION

The invention provides improved combination therapeutics and methods for the treatment of cancer in a mammal, typically a human, by administering a combination of an anti-Tyrosine kinase inhibitor and an antibody that specifically binds to human Insulin-Like Growth Factor receptor Type 1 (IGF-1R).

In one aspect of the invention, the tyrosine kinase inhibitor is Erlotinib.

In another aspect of the invention, the IGF-1R antibody is MK-0646, an anti-IGF-1R antibody.

In yet a further aspect of the invention, administration of the combination results in enhanced therapeutic efficacy relative to administration of the tyrosine kinase inhibitor alone.

In yet another aspect of the invention, the Tyrosine kinase inhibitor is typically administered orally, prior to, or concurrent with the administration of the IGF-1R antibody (MK-0646).

In another aspect of the invention, the anti-IGF-1R antibody may be administered prior to, at the same time as, or following administration of the tryrpsine kinase inhibitor. The anti-IGF-1R antibody may be administered via parenteral, e.g., subcutaneous, intratumoral, intravenous, intradermal, oral, transmucosal, or rectal administration. While not intending to be bound to a particular theory of operation, it is believed that blockade of IGF-1R mediated signaling cascade through the administration of an anti-IGF-1R antibody potentiates anti-tumor immunity by negatively modulating the signaling cascade attendant the binding of a native IGF-1R ligand to the receptor.

In yet another embodiment, the present invention provides a method for treating or preventing a medical condition, in a subject, comprising administering a therapeutically effective amount of an one or more IGF1R inhibitors or pharmaceutical compositions thereof to the subject. In an embodiment, the IGF1R inhibitor is selected from the group consisting of

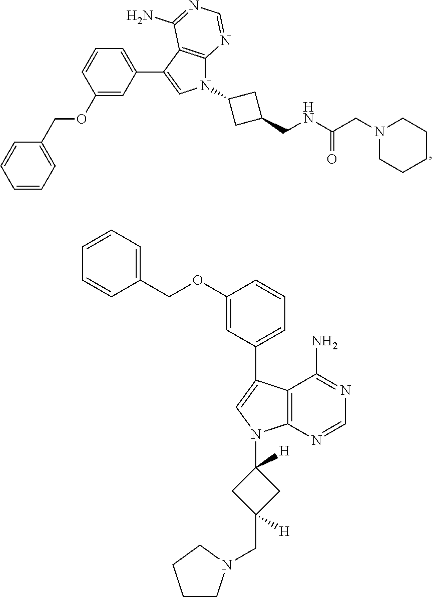

and an isolated antibody that binds specifically to IGF1R (e.g., human IGF1R) or an antigen-binding fragment thereof. In an embodiment, the antibody comprises Dalotuzumab or any other IGF1R inhibitor set forth herein, for example, under the “IGF1R inhibitors” section below. In an embodiment, the IGF1R inhibitor is administered in association with one or more further anti-cancer chemotherapeutic agents or a pharmaceutical composition thereof.

In an embodiment, the further anti-cancer chemotherapeutic agent is a member selected from the group consisting of teniposide

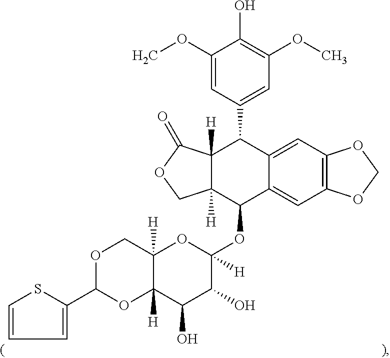

cisplatin

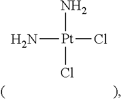

carboplatin

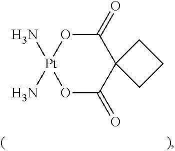

etoposide

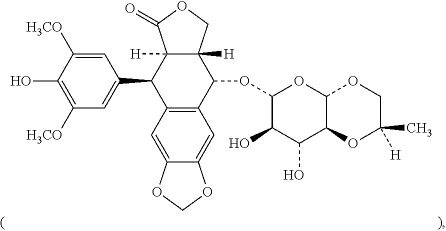

doxorubicin

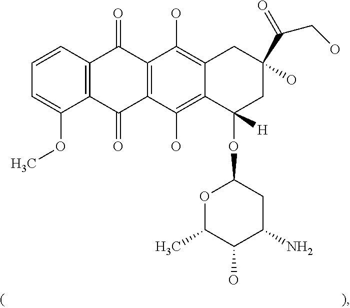

any liposomal formulation thereof such as Caelyx or Doxil®, cyclophosphamide

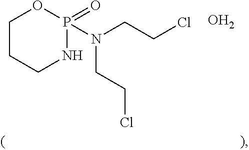

13-cis-retinoic acid

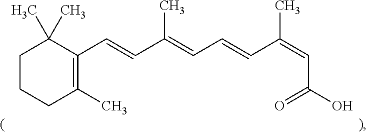

ifosfamide

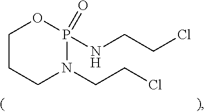

gemcitabine

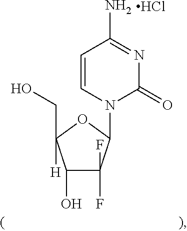

irinotecan

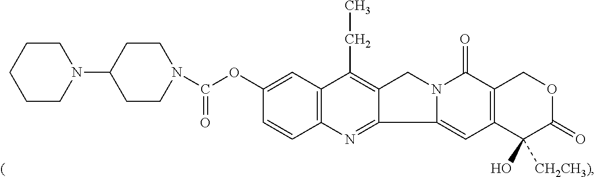

vincristine

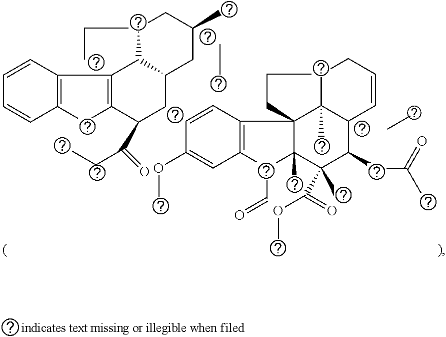

dactinomycin

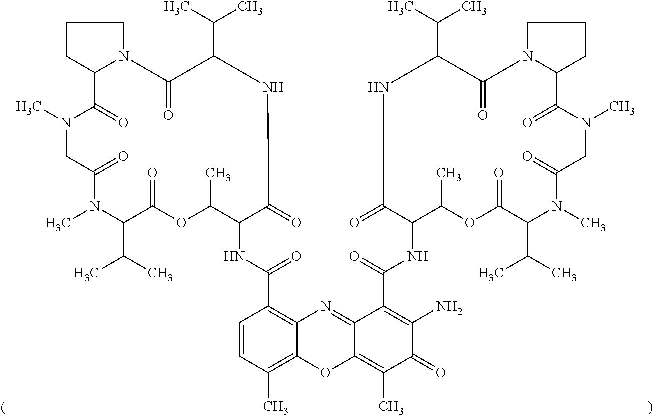

methotrexate

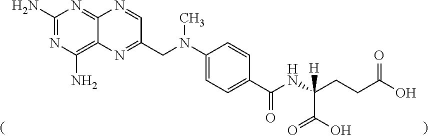

and any other chemotherapeutic agent set forth herein, for example, as set forth under the “Further Chemotherapeutics” section below. In an embodiment, the dosage of any anti-IGF1R antibody set forth herein is in the range of about 1-20 mg/kg of body weight or about 40-1000 mg/m². In an embodiment, the IGF1R inhibitor and the further anti-cancer therapeutic agent are administered simultaneously. In an embodiment, the IGF1R inhibitor and the further anti-cancer therapeutic agent are administered non-simultaneously. In an embodiment, the antibody comprises an IgG constant region. In an embodiment, the subject is a human (e.g., a child). In an embodiment, the IGF1R inhibitor is administered in association with an anti-cancer therapeutic procedure. In an embodiment, the anti-cancer therapeutic procedure is surgical tumorectomy and/or anti-cancer radiation treatment.

In an embodiment of the invention, the anti-IGF1R antibody or antigen-binding fragment thereof comprises one or more 2.12.1 fx CDRs (e.g., 3 light chain CDRs and/or 3 heavy chain CDRs) as set forth herein.

The invention further provides compositions and kits comprising an EGFR inhibitor(s) and an anti-IGF-1R antagonist for use according to the description provided herein.

The term “antibodies” as used herein includes monoclonal, polyclonal, chimeric, single chain, bispecific, and humanized or optimized antibodies as well as Fab fragments, such as those fragments which maintain the binding specificity of the antibodies to the IGF-1R proteins, including fragments thereof that express the same epitope as that bound by the antibodies of the invention.

Other characteristics and advantages of the invention appear in the continuation of the description with the examples and the figures whose legends are represented below.

### BRIEF DESCRIPTION OF THE DRAWINGS

FIG. 1 (A) is a Schematic showing cross talk between EGF1R & IGF1R (no method needed)

FIG. 1 (B) Summary of EGFR & IGF1R activation as measured by P-RTK array (Detailed method or cell culture, lysis, RTK array methods & image quatification are given in the document)

FIG. 1 (C) is representative images from the P-RTK array, positions corresponding to P-EGFR & P-IGF1R are indicated in the image (method is the same as in FIG. 1 B)

### DETAILED DESCRIPTION OF THE INVENTION

As a result of assiduous studies, the present inventors have found that a synergistically excellent anticancer activity can be achieved by using an an anti-IGF-1R antibody or a pharmaceutically acceptable salt thereof in combination with a tyrosine kinase inhibitor. The IGF-1R antibody is one of dalotuzumab, figitumumab, cixutumumab, SHC 717454, Roche R1507, EM164 or Amgen AMG479.

A broad aspect of the invention relates to a method of enhancing the anti-tumor response in a mammal. The invention is especially useful in the treatment of a cancer selected from the group consisting of non-small cell lung cancer, breast cancer, colorectal cancer, soft tissue or bone sarcomas and endometrial cancer. However, the instant invention could prove useful in the treatment of various other cancers, such as brain cancer, cervicocerebral cancer, esophageal cancer, thyroid cancer, small cell lung cancer, lung cancer, stomach cancer, gallbladder/bile duct cancer, liver cancer, pancreatic cancer, ovarian cancer, choriocarcinoma, uterus body cancer, uterocervical cancer, renal pelvis/ureter cancer, bladder cancer, prostate cancer, penis cancer, testicles cancer, fetal cancer, Wilms' cancer, skin cancer, malignant melanoma, neuroblastoma, osteosarcoma, Ewing's tumor, soft part sarcoma, acute leukemia, chronic lymphatic leukemia, chronic myelocytic leukemia and Hodgkin's lymphoma. More particularly, the invention is concerned with combinations comprising a tyrosine kinase inhibitor and an anti-IGF-1R antibody, and methods of administering the combination for treating NSCLC.

### Definitions and General Techniques

The reference works, patents, patent applications, and scientific literature, including accession numbers to GenBank database sequences that are referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise. Thus, for example, reference to “a genetic alteration” includes a plurality of such alterations and reference to “a probe” includes reference to one or more probes and equivalents thereof known to those skilled in the art, and so forth.

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis according to the present invention.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancerous” or “malignant cell” is understood as a cell having specific structural properties, lacking differentiation and being capable of invasion and metastasis. Examples of cancers are kidney, colon, breast, prostate and liver cancer. (see DeVita, V. et al. (eds.), 2001, Cancer Principles And Practice Of Oncology, 6.sup.th Ed., Lippincott Williams & Wilkins, Philadelphia, Pa.; this reference is herein incorporated by reference in its entirety for all purposes). More specifically, while the examples detail the treatment of NSCLC using the combination therapeutic detailed herein, the term “cancer” is not so limited. It includes any and all tumours that are IGF-1R dependent as well as EGFR-dependent. Exemplary cancers if this type includes for example pancreatic cancer.

A feature of cancer cells is the tendency to grow in a manner that is uncontrollable by the host, but the pathology associated with a particular cancer cell may take any form. Primary cancer cells (that is, cells obtained from near the site of malignant transformation) can be readily distinguished from non-cancerous cells by well-established pathology techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells.

Cell line—A “cell line” or “cell culture” denotes higher eukaryotic cells grown or maintained in vitro. It is understood that the descendants of a cell may not be completely identical (either morphologically, genotypically, or phenotypically) to the parent cell. Cells described as “uncultured” are obtained directly from a living organism, and have been maintained for a limited amount of time away from the organism: not long enough or under conditions for the cells to undergo substantial replication.

“Diagnosing” a disease as used in the application is intended to include, for example, diagnosing or detecting the presence of a pathological hyperproliferative oncogenic disorder associated with or mediated by expression of IGF-1R, monitoring the progression of the disease, and identifying or detecting cells or samples that are indicative of a disorder associated with expression of IGF-1R. The terms diagnosing, detecting, identifying etc. are used interchangeably herein.

“Pathology” as used herein—The “pathology” caused by cancer cells within a host is anything that compromises the well-being or normal physiology of the host. This may involve, but is not limited to abnormal or uncontrollable growth of the cancer cell, metastasis, increase in expression levels of IGF-1R bearing cells, or other products at an inappropriate level, manifestation of a function inappropriate for its physiological milieu, interference with the normal function of neighboring cells, aggravation or suppression of an inflammatory or immunological response, or the harboring of undesirable chemical agents or invasive organisms.

“Treatment” of an individual or a cell is any type of intervention in an attempt to alter the non-treated course of the individual or cell. For example, treatment of an individual may be undertaken to decrease or limit the pathology caused by a cancer harbored in the individual. Treatment includes but is not limited to a) administration of a composition or a combination therapeutic, such as a pharmaceutical composition comprising an IGF-1R specific mAb and a tyrosine kinase inhibitor. The term “treating” refers to having a therapeutic effect and at least partially alleviating or abrogating an abnormal condition in the organism. Treating includes inhibition of tumor growth, maintenance of inhibited tumor growth, and induction of remission.

The term “preventing” refers to decreasing the probability that an organism contracts or develops an abnormal condition.

As used herein, the term “about” refers to an approximation of a stated value within an acceptable range. Preferably the range is +/−5% of the stated value.

The term “or” is used herein to mean, and is used interchangeably with, the term “and/or”, unless context clearly indicates otherwise.

The terms “IGF1R”, “IGFR1”, “Insulin-like Growth Factor Receptor-I” and “Insulin-like Growth Factor Receptor, type I” are well known in the art. Although IGF-1R may be from any organism, it is preferably from an animal, more preferably from a mammal (e.g., mouse, rat, rabbit, sheep or dog) and most preferably from a human. The nucleotide and amino acid sequence of a typical human IGF-1R precursor is available at Genbank, eg. Gene ID 3480 or NM000875. Cleavage of the precursor (e.g., between amino acids 710 and 711) produces an α-subunit and a β-subunit which associate to form a mature receptor.

An “immunoglobulin” is a tetrameric molecule. In a naturally-occurring immunoglobulin, each tetramer is composed of two identical pairs of polypeptide chains, each pair having one “light” (about 25 kDa) and one “heavy” chain (about 50 70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as .kappa. and .lamda. light chains. Heavy chains are classified as .mu., .DELTA., .gamma., .alpha., or .epsilon., and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

An “antibody” refers to an intact immunoglobulin or to an antigen-binding portion thereof that competes with the intact antibody for specific binding. Antigen-binding portions may be produced by recombinant DNA techniques or by enzymatic or chemical cleavage of intact antibodies. Antigen-binding portions include, inter alia, Fab, Fab′, F(ab′)2, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide. There are several anti-IGF1R antibodies that are known in the art (see e.g., WO 03/100008; WO 2002/53596; WO 04/71529; WO 03/106621; US2003/235582; WO 04/83248; WO 03/59951; WO 04/87756 or WO 2005/16970). Other small molecule IGF1R inhibitors are also known in the art

As used in the application, the term “anti-IGF-1R antibody” is collectively referred to as an anti-IGF-1R antibody disclosed in U.S. Pat. No. 7,241,444, filed Dec. 16, 2003, the entire content of which is incorporated by reference herein in its entirety. The amino acid sequences of the various CDRs, light and heavy chain as well as the nucleotide sequences encoding the entire antibody claimed therein area also incorporated in their entirety by reference herein. Likewise, the disclosure of Ser. No. 11/801,080 is also incorporated by reference herein in its entirety.

The term “patient” includes human and veterinary subjects.

Antibodies—IGF-1R (h7C 10)

As detailed herein, an aspect of the present invention is directed to a method of improving the anti-tumor efficacy of an anti-cancer agent by co-administering a tyrosine kinase inhibitor—EGFR, e.g., erlotinib and an antibody which specifically binds to human Insulin-like growth factor-1 receptor (IGF-1R)-1 to a patient with cancer.

As a consequence, the IGF-1R antibody for use in the proposed combination therapeutic is one that specifically bind insulin-like growth factor 1 receptor (IGF-1R). Exemplary anti-IGF-1R antibodies for use in the combination therapeutic and methods for of use thereof are described in U.S. Pat. No. 7,241,444 ('444 patent) the content of which is incorporated by reference herein in its entirety. See for example Claim 1 of the '444 patent. “h7C10” or “MK-0646” are used interchangeably to describe a humanized antibody that is characterized as binding IGF-1R as well as binding the IR/IGF-1 hybrid receptor. Such an antibody preferably includes the antibody described, for example, in the '444 patent, wherein the antibody is a humanized antibody or a fragment thereof and comprises a light chain and/or a heavy chain in which the skeleton segments FR1 to FR4 of said light chain and/or heavy chain are respectively derived from skeleton segments FR1 to FR4 of human antibody light chain and/or heavy chain. The humanized antibody may comprise at least one light chain that comprises at least one or more complementary determining regions derived from a non-human source and having the amino acid sequence selected from the group consisting of SEQ ID NOs: 1, 2, or 3 and at least one heavy chain comprising at least one or more complementary determining regions having an amino acid sequence selected from the group consisting of SEQ ID NOs 4, 5 or 6. The light chain may comprise one or more of the amino acid sequences as set forth in one of SEQ ID NOs. 7 or 8, or a sequence having at least 80% identity after optimum alignment with the sequence SEQ ID Nos: 7 or 8. Likewise, the heavy chain comprises one or more amino acid sequences as set forth in one of SEQ ID No. 9, 10 03 11, or a sequence having at least 80% identity after optimum alignment with the sequence SEQ ID Nos 9, 10 or 11. In certain embodiments, the methods of treatment include administering an antibody that binds the same epitope on IGF-1R as that bound by MK-0646.

Nucleic acid molecule for expressing the recombinant antibodies (IGF-1R specific mAbs) are described in the '444 patent, the content of which is incorporated by reference herein in its entirety

“Nucleic acid” or a “nucleic acid molecule” as used herein refers to any DNA or RNA molecule, either single- or double-stranded and, if single-stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. In some embodiments of the invention, nucleic acids are “isolated.” This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

Nucleic acids of the invention also include fragments of the nucleic acids of the invention. A “fragment” refers to a nucleic acid sequence that is preferably at least about 10 nucleic acids in length, more preferably about 40 nucleic acids, and most preferably about 100 nucleic acids in length. A “fragment” can also mean a stretch of at least about 100 consecutive nucleotides that contains one or more deletions, insertions, or substitutions. A “fragment” can also mean the whole coding sequence of a gene and may include 5′ and 3′ untranslated regions.

The antibodies for use in the present invention include, but are not limited to, monoclonal antibodies, synthetic antibodies, polyclonal antibodies, multispecific antibodies (including bi-specific antibodies), human antibodies, humanized antibodies, chimeric antibodies, single-chain Fvs (scfv) (including bi-specific scFvs), single chain antibodies, Fab fragments, F(ab′) fragments, disulfide-linked Fvs (sdFv), and epitope-binding fragments of any of the above. In particular, antibodies for use in the present invention include immunoglobulin molecules and immunologically active portions of immunoglobulin molecules, i.e., molecules that contain a IGF-1R binding site that immunospecifically binds to IGF-1R. The immunoglobulin molecules for use in the invention can be of any type (e.g. IgG, IgE, IgM, IgD, IgA and IgY), class (e.g., IgG1, IgG2, IgG3, IgG4, IgA1 and IgA2) or subclass of immunoglobulin molecule. Preferably, the antibodies for use in the invention are IgG, more preferably, IgG1.

The antibodies for use in the invention may be from any animal origin. Preferably, the antibodies are humanized monoclonal antibodies. Alternatively, to antibodies may be fully human so long as they bind the same epitope of the antibody claimed in the '444 patent. As used herein, “human” antibodies include antibodies having the amino acid sequence of a human immunoglobulin and include antibodies isolated from human immunoglobulin libraries or from mice or other animals that express antibodies from human genes.

The antibodies for use in the present invention may be monospecific, bispecific, trispecific or of greater multispecificity. Multispecific antibodies may immunospecifically bind to different epitopes of a polypeptide or may immunospecifically bind to both a polypeptide as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

The antibodies for use in the invention include derivatives of the antibodies. Standard techniques known to those of skill in the art can be used to introduce mutations in the nucleotide sequence encoding an antibody to be used with the methods for use in the invention, including, for example, site-directed mutagenesis and PCR-mediated mutagenesis which result in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

The antibodies for use in the present invention include derivatives that are modified, i.e., by the covalent attachment of any type of molecule to the antibody. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, synthesis in the presence of tunicamycin, etc. Additionally, the derivative may contain one or more non-classical amino acids.

The present invention also provides antibodies for use in the invention that comprise a framework region known to those of skill in the art. In certain embodiments, one or more framework regions, preferably, all of the framework regions, of an antibody to be used in the compositions and methods for use in the invention are human. In certain other embodiments for use in the invention, the fragment region of an antibody for use in the invention is humanized. In certain embodiments, the antibody to be used with the methods for use in the invention is a synthetic antibody, a monoclonal antibody, an intrabody, a chimeric antibody, a human antibody, a humanized chimeric antibody, a humanized antibody, a glycosylated antibody, a multispecific antibody, a human antibody, a single-chain antibody, or a bispecific antibody.

In certain embodiments, an antibody for use in the invention has a high binding affinity for IGF-1R.

In certain embodiments, an antibody for use in the invention has a half-life in a subject, preferably a human, of about 12 hours or more, about 1 day or more, about 3 days or more, about 6 days or more, about 10 days or more, about 15 days or more, about 20 days or more, about 25 days or more, about 30 days or more, about 35 days or more, about 40 days or more, about 45 days or more, about 2 months or more, about 3 months or more, about 4 months or more, or about 5 months or more. Antibodies with increased in vivo half-lives can be generated by techniques known to those of skill in the art. For example, antibodies with increased in vivo half-lives can be generated by modifying (e.g., substituting, deleting or adding) amino acid residues identified as involved in the interaction between the Fc domain and the FcRn receptor (see, e.g., International Publication No. WO 97/34631 and U.S. patent application Ser. No. 10/020,354, entitled “Molecules with Extended Half-Lives, Compositions and Uses Thereof”, filed Dec. 12, 2001, by Johnson et al.; and U.S. Publication Nos. 2005/003700 and 2005/0064514, which are incorporated herein by reference in their entireties). Such antibodies can be tested for binding activity to antigens as well as for in vivo efficacy using methods known to those skilled in the art, for example, by immunoassays described herein.

Further, antibodies with increased in vivo half-lives can be generated by attaching to the antibodies polymer molecules such as high molecular weight polyethyleneglycol (PEG). PEG can be attached to the antibodies with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography. PEG-derivatized antibodies can be tested for binding activity to antigens as well as for in vivo efficacy using methods known to those skilled in the art, for example, by immunoassays described herein.

In certain embodiments, an antibody for use in the present invention includes antigen-binding portions of an intact antibody that retain capacity to bind IGF-1R. Examples include (i) a Fab fragment, a monovalent fragment consisting of the VL, VH, CL and CH1 domains; (ii) a F(ab′)2 fragment, ambivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al. (1988) Science 242:423-426; and Huston et al. (1988) Proc. Natl. Acad Sci. USA 85:5879-5883). Such single chain antibodies are included by reference to the term “antibody.”

Methods of Producing Antibodies to IGF-1R are well known. See for example, the '444 patent.

Screening for Antibody Specificity—Techniques for generating antibodies have been described above. One may further select antibodies with certain biological characteristics, as desired. Thus, once produced, the antibodies may be screened for their binding affinity for IGF-1R. Screening for antibodies that specifically bind to IGF-1R may be accomplished using an enzyme-linked immunosorbent assay (ELISA) in which microtiter plates are coated with IGF-1R. In some embodiments, antibodies that bind IGF-1R from positively reacting clones can be further screened for reactivity in an ELISA-based assay to other IGF-1R isoforms, for example, IGF-1R using microtiter plates coated with the other IGF-1R isoform(s). Clones that produce antibodies that are reactive to another isoform of IGF-1R are eliminated, and clones that produce antibodies that are reactive to IGF-1R only may be selected for further expansion and development. Confirmation of reactivity of the antibodies to IGF-1R may be accomplished, for example, using a Western Blot assay in which protein from ovarian, breast, renal, colorectal, lung, endometrial, or brain cancer cells and purified IGF-1R and other IGF-1R isoforms are run on an SDS-PAGE gel, and subsequently are blotted onto a membrane. The membrane may then be probed with the putative anti-IGF-1R antibodies. Reactivity with IGF-1R and not another insulin-like receptor isoform confirms specificity of reactivity for IGF-1R.

General methods for detecting IGF-1R or its Derivatives—The assaying method for detecting IGF-1R using the antibodies of the invention or binding fragments thereof are not particularly limited. Any assaying method can be used, so long as the amount of antibody, antigen or antibody-antigen complex corresponding to the amount of antigen (e.g., the level of IGF-1R) in a fluid to be tested can be detected by chemical or physical means and the amount of the antigen can be calculated from a standard curve prepared from standard solutions containing known amounts of the antigen. Representative immunoassays encompassed by the present invention include, but are not limited to, those described in U.S. Pat. No. 4,367,110 (double monoclonal antibody sandwich assay); Wide et al., Kirkham and Hunter, eds. Radioimmunoassay Methods, E. and S. Livingstone, Edinburgh (1970); U.S. Pat. No. 4,452,901 (western blot); Brown et al., J. Biol. Chem. 255: 4980-4983 (1980) (immunoprecipitation of labeled ligand); and Brooks et al., Clin. Exp. Immunol. 39:477 (1980) (immunocytochemistry); immunofluorescence techniques employing a fluorescently labeled antibody, coupled with light microscopic, flow cytometric, or fluorometric detection etc. See also Immunoassays for the 80's, A. Voller et al., eds., University Park, 1981, Zola, Monoclonal Antibodies: A Manual of Techniques, pp. 147-158 (CRC Press, Inc. 1987).

(1) Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus funning an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

In the sandwich assay, the immobilized antibody of the present invention is reacted with a test fluid (primary reaction), then with a labeled form of antibody of the present invention (secondary reaction), and the activity of the labeling agent on the immobilizing carrier is measured, whereby the IGF-1R level in the test fluid can be quantified. The primary and secondary reactions may be performed simultaneously or with some time intervals. The methods of labeling and immobilization can be performed by modifications of those methods described above. In the immunoassay by the sandwich assay, the antibody used for immobilized or labeled antibody is not necessarily from one species, but a mixture of two or more species of antibodies may be used to increase the measurement sensitivity, etc. In the method of assaying IGF-1R by the sandwich assay, for example, when the antibodies used in the primary reaction recognize the partial peptides at the C-terminal region of IGF-1R, the antibodies used in the secondary reaction are preferably those recognizing partial peptides other than the C-terminal region (i.e., the N-terminal region). When the antibodies used for the primary reaction recognize partial peptides at the N-terminal region of IGF-1R, the antibodies used in the secondary reaction, antibodies recognizing partial peptides other than the N-terminal region (i.e., the C-terminal region) are preferably employed.

Other types of “sandwich” assays, which can also be useful for detecting IGF-1R, are the so-called “simultaneous” and “reverse” assays. A simultaneous assay involves a single incubation step wherein the antibody bound to the solid support and labeled antibody are both added to the sample being tested at the same time. After the incubation is completed, the solid support is washed to remove the residue of fluid sample and uncomplexed labeled antibody. The presence of labeled antibody associated with the solid support is then determined as it would be in a conventional “forward” sandwich assay.

In the “reverse” assay, stepwise addition first of a solution of labeled antibody to the fluid sample followed by the addition of unlabeled antibody bound to a solid support after a suitable incubation period, is utilized. After a second incubation, the solid phase is washed in conventional fashion to free it of the residue of the sample being tested and the solution of unreacted labeled antibody. The determination of labeled antibody associated with a solid support is then determined as in the “simultaneous” and “forward” assays. In one embodiment, a combination of antibodies of the present invention specific for separate epitopes can be used to construct a sensitive three-site immunoradiometric assay.

This type of assays may also be used to quantify IGF-1R expression in whatever “sample” it may present itself. Thus, in certain aspects, the sandwich assay includes:

(i) a method for quantifying expression levels of IGF-1R in a test fluid, comprising reacting the antibody specifically reacting with a partial peptide at the N-terminal region of the IGF-1R immobilized on a carrier, a labeled form of the antibody specifically reacting with a partial peptide at the C-terminal region and the test fluid, and measuring the activity of the label; or

(ii) a method for quantifying IGF-1R expression in a test fluid, comprising reacting the antibody specifically reacting with a partial peptide at the C-terminal region of the IGF-1R immobilized onto a carrier, the antibody specifically reacting with a partial peptide at the N-terminal region of a labeled form of the IGF-1R and the test fluid, and measuring the activity of the label; etc.

(2) Competitive binding assays rely on the ability of a labeled standard to compete with the test sample analyte for binding with a limited amount of antibody. The amount of IGF-1R protein in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

For quantifying the level of IGF-1R expression, one skilled in the art may combine and/or competitively react antibodies of the invention or fragments thereof, a test fluid and a labeled form of IGF-1R, measure a ratio of the labeled IGF-1R bound to the antibodies or fragments thereof b to thereby quantify the IGF-1R in the test fluid.

(3) Immunometric Assay

In the immunometric assay, an antigen in a test fluid and a solid phase antigen are competitively reacted with a given amount of a labeled form of the antibody of the present invention followed by separating the solid phase from the liquid phase; or an antigen in a test fluid and an excess amount of labeled form of the antibody of the present invention are reacted, then a solid phase antigen is added to bind an unreacted labeled form of the antibody of the present invention to the solid phase and the solid phase is then separated from the liquid phase. Thereafter, the labeled amount of any of the phases is measured to determine the antigen level in the test fluid.

Typical, and preferred, immunometric assays include “forward” assays in which the antibody bound to the solid phase is first contacted with the sample being tested to extract the IGF-1R from the sample by formation of a binary solid phase antibody-IGF-1R complex. After a suitable incubation period, the solid support is washed to remove the residue of the fluid sample, including unreacted IGF-1R, if any, and then contacted with the solution containing a known quantity of labeled antibody (which functions as a “reporter molecule”). After a second incubation period to permit the labeled antibody to complex with the IGF-1R bound to the solid support through the unlabeled antibody, the solid support is washed a second time to remove the unreacted labeled antibody. This type of forward sandwich assay can be a simple “yes/no” assay to determine whether IGF-1R is present or can be made quantitative by comparing the measure of labeled antibody with that obtained for a standard sample containing known quantities of IGF-1R. Such “two-site” or “sandwich” assays are described by Wide (Radioimmune Assay Method, Kirkham, ed., Livingstone, Edinburgh, 1970, pp. 199-206).

(4) Nephrometry

In the nephrometry, the amount of insoluble sediment, which is produced as a result of the antigen-antibody reaction in a gel or in a solution, is measured. Even when the amount of an antigen in a test fluid is small and only a small amount of the sediment is obtained, a laser nephrometry utilizing laser scattering can be suitably used.

Examples of labeling agents, which may be used in the above referenced assay methods (1) to (4) using labeling agents, include radioisotopes (e.g., 125I, 131I, 3H, 14C, 32P, 33P, 35S, etc., fluorescent substances, e.g., cyanine fluorescent dyes (e.g., Cy2, Cy3, Cy5, Cy5.5, Cy7), fluorescamine, fluorescein isothiocyanate, etc., enzymes (e.g., .beta.-galactosidase, .beta.-glucosidase, alkaline phosphatase, peroxidase, malate dehydrogenase, etc.), luminescent substances (e.g., luminol, a luminol derivative, luciferin, lucigenin, etc.), biotin, lanthanides, etc. In addition, a biotin-avidin system may be used as well for binding an antibody to a labeling agent.

In the immobilization of antigens or antibodies, physical adsorption may be used. Alternatively, chemical binding that is conventionally used for immobilization of proteins, enzymes, etc. may be used as well. Examples of the carrier include insoluble polysaccharides such as agarose, dextran, cellulose, etc.; synthetic resins such as polystyrene, polyacrylamide, silicone, etc.; or glass; and the like.

In another embodiment, the present invention assists in the diagnosis of cancers and tumors by the identification and measurement of the IGF-1R levels in body fluids, such as blood, serum, plasma, sputum and the like. If IGF-1R is normally present, and the development of the oncogenic disorder is caused by an abnormal quantity of the cell surface receptor (IGF-1R), e.g., expression relative to normal, the assay should compare IGF-1R levels in the biological sample to the range expected in normal, non-oncogenic tissue of the same cell type. Thus, a statistically significant increase in the amount of IGF-1R bearing cells or IGF-1R expression level in the subject relative to the control subject or subject's baseline, can be a factor that may lead to a diagnosis of an oncogenic disorder that is progressing or at risk for such a disorder. Likewise, the presence of high levels of IGF-1R indicative of cancers likely to metastasize can also be detected. For those cancers that express the antigen recognized by the antibodies of the invention, e.g., IGF-1R, the ability to detect the antigen provides early diagnosis, thereby affording the opportunity for early treatment. Early detection is especially important for cancers difficult to diagnose in their early stages.

Moreover, the level of antigen detected and measured in a body fluid sample such as blood provides a means for monitoring the course of therapy for the cancer or tumor, including, but not limited to, surgery, chemotherapy, radiation therapy, the therapeutic methods of the present invention, and combinations thereof. By correlating the level of the antigen in the body fluid with the severity of disease, the level of such antigen can be used to indicate successful removal of the primary tumor, cancer, and/or metastases, for example, as well as to indicate and/or monitor the effectiveness of other therapies over time. For example, a decrease in the level of the cancer or tumor-specific antigen over time indicates a reduced tumor burden in the patient. By contrast, no change, or an increase, in the level of antigen over time indicates ineffectiveness of therapy, or the continued growth of the tumor or cancer.

Detection of the antibody in the specimen can be accomplished using techniques known in the art such as immunoenzymatic techniques, e.g., immunoperoxidase staining technique, or the avidin-biotin technique, or immunofluorescence techniques (see, e.g., Ciocca et al., 1986, “Immunohistochemical Techniques Using Monoclonal Antibodies”, Meth. Enzymol., 121:562 79 and Introduction to Immunology, Ed. Kimball, (2.sup.nd Ed), Macmillan Publishing Company, 1986, pp. 113 117). Those skilled in the art can determine operative and optimal assay conditions by routine experimentation.

A typical in vitro immunoassay for detecting IGF-1R comprises incubating a biological sample in the presence of a detectably labeled anti-IGF-1R antibody or antigen binding fragment of the present invention capable of selectively binding to IGF-1R, and detecting the labeled fragment or antibody which is bound in a sample. The antibody is bound to a label effective to permit detection of the cells or portions (e.g., IGF-1R or fragments thereof liberated from hyperplastic, dysplastic and/or cancerous cells) thereof upon binding of the antibody to the cells or portions thereof. The presence of any cells or portions thereof in the biological sample is detected by detection of the label.

The biological sample may be brought into contact with, and immobilized onto, a solid phase support or carrier, such as nitrocellulose, or other solid support or matrix, which is capable of immobilizing cells, cell particles, membranes, or soluble proteins. The support may then be washed with suitable buffers, followed by treatment with the detectably-labeled anti-IGF-1R antibody. The solid phase support may then be washed with buffer a second time to remove unbound antibody. The amount of bound label on the solid support may then be detected by conventional means. Accordingly, in another embodiment of the present invention, compositions are provided comprising the monoclonal antibodies, or binding fragments thereof, bound to a solid phase support, such as described herein.

By “solid phase support” or “carrier” is intended any support capable of binding peptide, antigen or antibody. Well-known supports or carriers, include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention. The support material can have virtually any possible structural configuration so long as the coupled molecule is capable of binding to IGF-1R or an Anti-IGF-1R antibody. Thus, the support configuration can be spherical, as in a bead, or cylindrical, as in the inside surface of a test tube, or the external surface of a rod. Alternatively, the surface can be flat, such as a sheet, culture dish, test strip, etc. Preferred supports include polystyrene beads. Those skilled in the art will know many other suitable carriers for binding antibody, peptide or antigen, or can ascertain the same by routine experimentation.

In vitro assays in accordance with the present invention also include the use of isolated membranes from cells expressing a recombinant IGF-1R, soluble fragments comprising the ligand binding segments of IGF-1R, or fragments attached to solid phase substrates. These assays allow for the diagnostic determination of the effects of either binding segment mutations and modifications, or ligand mutations and modifications, e.g., ligand analogues.

Assays For Efficacy of Combination Immunotherapy in In vivo Models—Tumor burden can be assessed at various time points after tumor challenge using techniques well known in the art. Assays for monitoring anti-tumor response and determining the efficacy of combination immunotherapy are described below. While an improved or enhanced anti-tumor response may be most dramatically observed shortly following administration of the immunotherapy, e.g. within 5-10 days, the response may be delayed in some instances, depending on factors such as the expression level of the IGF-1R, the dosage and dosing frequency of the anti-IGF-1R antibody, and the relative timing of administration of the anti-IGF-1R-1 antibody relative to the timing of administration of the tyrosine kinase inhibitor—Erlotinib. Thus, any of the well known assays may be performed on biological samples harvested at various time points following treatment or administration of the combination therapeutic in order to fully assess the anti-tumor response following immunotherapy.

Monitoring Treatment—One skilled in the art is aware of means to monitor the therapeutic outcome and/or the systemic immune response upon administering a combination treatment of the present invention. In particular, the therapeutic outcome can be assessed by monitoring attenuation of tumor growth and/or tumor regression and or the level of tumor specific markers. The attenuation of tumor growth or tumor regression in response to treatment can be monitored using one or more of several end-points known to those skilled in the art including, for instance, number of tumors, tumor mass or size, or reduction/prevention of metastasis.

IGF-1R inhibitors:

In an embodiment of the invention, an IGF1R inhibitor is BMS-577098

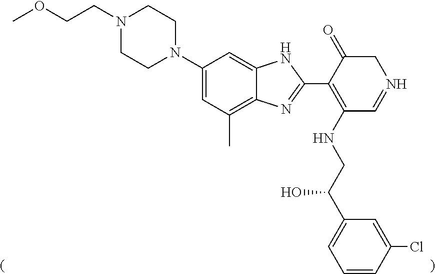

### or AEW-541

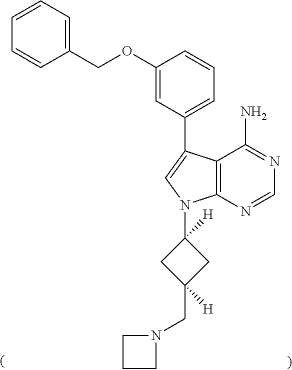

or

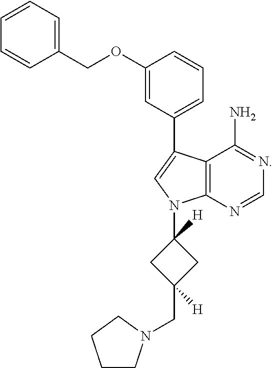

In an embodiment of the invention, an IGF1R inhibitor is any of the pyrimidine derivatives set forth in WO 03/48133, for example comprising the core structure:

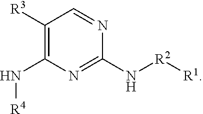

Methods of treating or preventing an Erlotinib resistant cancer or one mediated by IGF-1R by administering these agents are within the scope of the present invention.

In an embodiment of the invention, an IGF1R inhibitor is any of the tyrosine kinase inhibitors set forth in WO 03/35614, for example comprising the core structure:

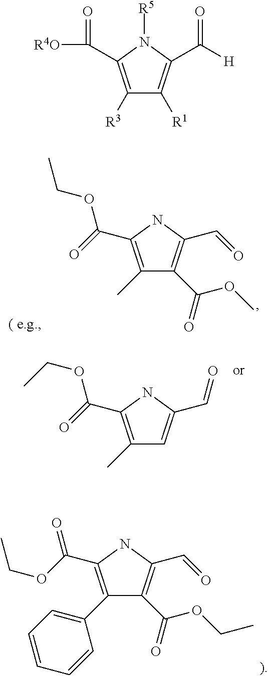

In an embodiment of the invention, an IGF1R inhibitor is any of the tyrosine kinase inhibitors set forth in WO 03/35615, for example comprising the core structure:

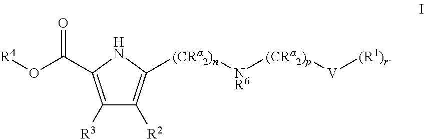

In an embodiment of the invention, an IGF1R inhibitor is any of the tyrosine kinase inhibitors set forth in WO 03/35616, for example comprising the core structure:

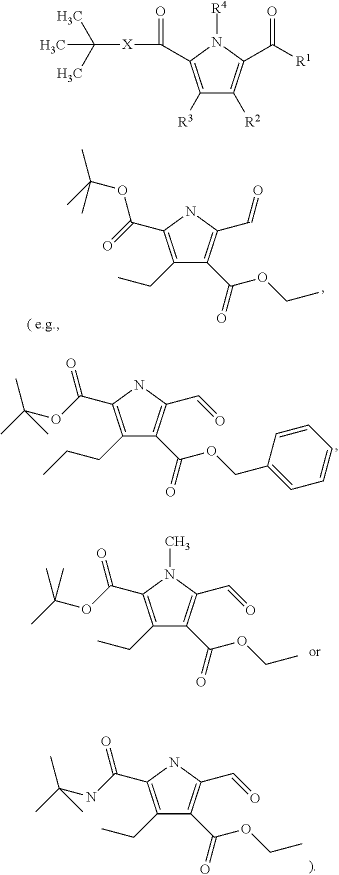

In an embodiment of the invention, an IGF1R inhibitor is any of the tyrosine kinase inhibitors set forth in WO 03/35619, for example comprising the core structure:

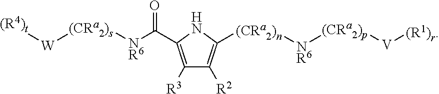

In an embodiment of the invention, an IGF1R inhibitor is a multitargeted kinase inhibitor which also inhibits e.g., VEGF-2R, Kit, FLT3 and/or PDGFR, for example, SU-11248 (e.g., sunitinib malate) or Bay43-9006 (sorafenib). Methods of treating or preventing an Erlotinib resistant cancer or one mediated by IGF-1R by administering these agents is within the scope of the present invention.

In an embodiment of the invention, an IGF1R inhibitor is any of the compounds set forth in WO 03/24967, for example comprising the core structure:

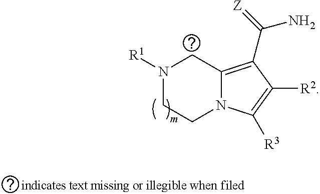

In an embodiment of the invention, an IGF1R inhibitor is any of the compounds set forth in WO 04/30625, for example comprising the core structure:

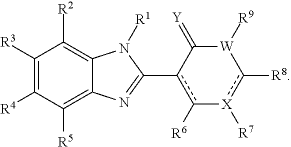

In an embodiment of the invention, an IGF1R inhibitor is any of the compounds set forth in WO 04/30627, for example comprising the core structure:

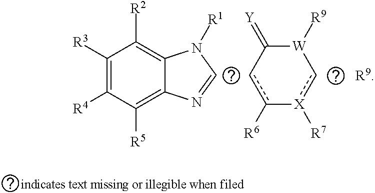

In an embodiment of the invention, an IGF1R inhibitor is any of the heteroaryl-aryl ureas set forth in WO 00/35455, for example comprising the core structure:

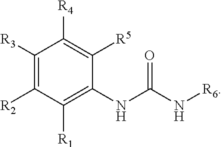

In an embodiment of the invention, an IGF1R inhibitor is any of the peptides set forth in WO 03/27246.

In an embodiment of the invention, an IGF1R inhibitor is

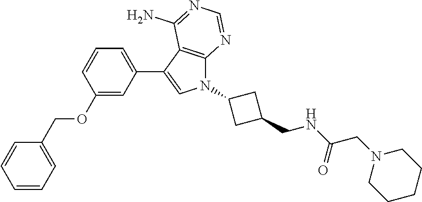

or any 4-amino-5-phenyl-7-cyclobutyl-pyrrolo[2,3-d] pyrimidine derivative disclosed in PCT Application Publication No. WO 02/92599.

### Further Chemotherapeutics

The scope of the present invention comprises compositions comprising an IGF1R inhibitor of the invention in association with a further chemotherapeutic agent along with methods for treating neuroblastoma, Wilm's tumor, osteosarcoma, rhabdomyosarcoma, pediatric cancers or pancreatic cancer by administering the IGF1R inhibitor in association with the further chemotherapeutic agent (e.g., a further anti-cancer chemotherapeutic agent or anti-emetic). A further chemotherapeutic agent comprises any agent that elicits a beneficial physiological response in an individual to which it is administered; for example, wherein the agent alleviates or eliminates disease symptoms or causes within the subject to which it is administered. A further chemotherapeutic agent includes any anti-cancer chemotherapeutic agent. An anti-cancer therapeutic agent is any agent that, for example, agent alleviates or eliminates symptoms or causes of cancer in the subject to which it is administered.

In an embodiment of the invention, an IGF1R inhibitor is provided in association with etoposide (VP-16;

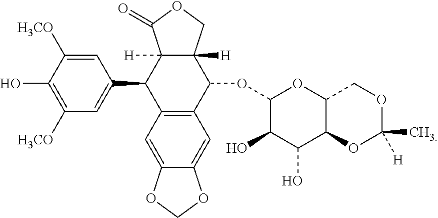

Methods of treating or preventing rhabdomyosarcoma, Wilm's tumor, osteosarcoma, neuroblastoma, pancreatic cancer, or any pediatric cancer by administering these agents are within the scope of the present invention.

In an embodiment of the invention, an IGF1R inhibitor is provided in association with gemcitabine

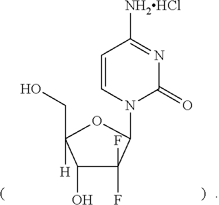

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with any compound disclosed in published U.S. patent application no. U.S. 2004/0209878A1 (e.g., comprising a core structure represented by

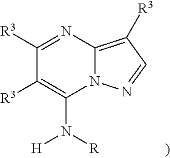

or doxorubicin

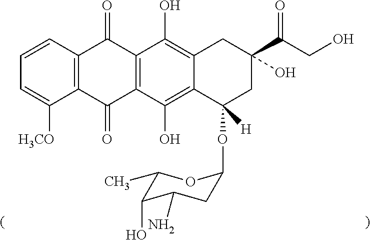

including Caelyx or Doxil® (doxorubicin HCl liposome injection; Ortho Biotech Products L.P; Raritan, N.J.). Doxil® comprises doxorubicin in STEALTH® liposome carriers which are composed of N-(carbonyl-methoxypolyethylene glycol 2000)-1,2-distearoyl-sn-glycero-3-phosphoethanolamine sodium salt (MPEG-DSPE); fully hydrogenated soy phosphatidylcholine (HSPC), and cholesterol. Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with 5′-deoxy-5-fluorouridine

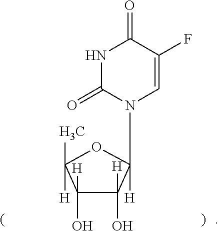

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with vincristine

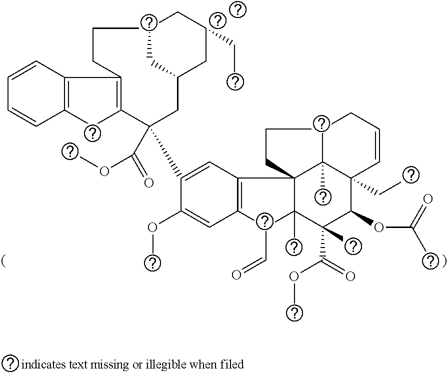

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with temozolomide

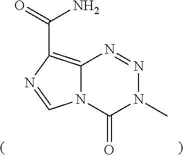

any CDK inhibitor such as ZK-304709, Seliciclib (R-roscovitine)

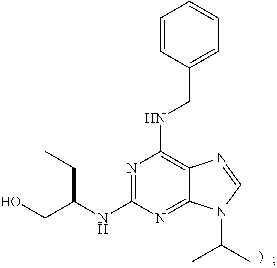

any MEK inhibitor such as PD0325901

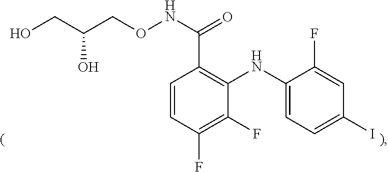

AZD-6244; capecitabine (5′-deoxy-5-fluoro-N-[(pentyloxy) carbonyl]-cytidine); or L-Glutamic acid, N-[4-[2-(2-amino-4,7-dihydro-4-oxo-1 H-pyrrolo[2,3-d]pyrimidin-5-yl)ethyl]benzoyl], disodium salt, heptahydrate

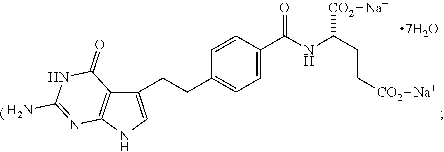

Pemetrexed disodium heptahydrate). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with camptothecin

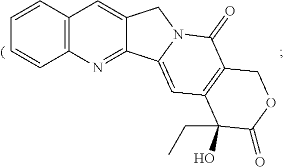

Stork et al., J. Am. Chem. Soc. 93 (16): 4074-4075 (1971); Beisler et al., J. Med. Chem. 14 (11): 1116-1117 (1962)) or irinotecan (

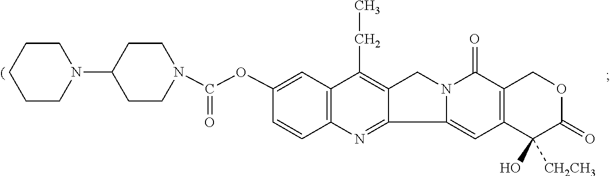

sold as Camptosar®; Pharmacia & Upjohn Co.; Kalamazoo, Mich.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with the FOLFOX regimen (oxaliplatin

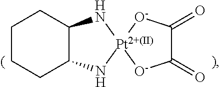

together with infusional fluorouracil

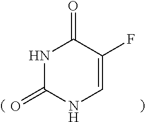

and folinic acid

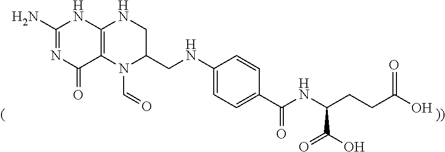

(Chaouche et al., Am. J. Clin. Oncol. 23 (3):288-289 (2000); de Gramont et al., J. Clin. Oncol. 18 (16):2938-2947 (2000)). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with an antiestrogen such as

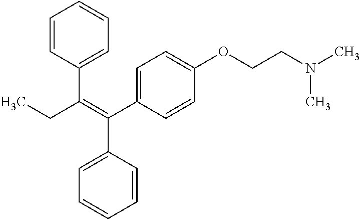

(tamoxifen; sold as Nolvadex® by AstraZeneca Pharmaceuticals LP; Wilmington, Del.) or

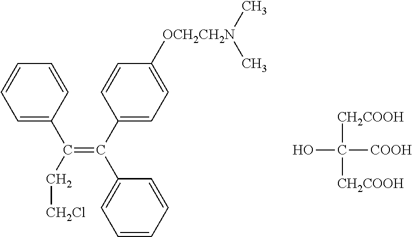

(toremifene citrate; sold as Fareston® by Shire US, Inc.; Florence, Ky.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with an aromatase inhibitor such as

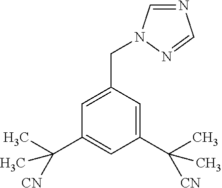

(anastrazole; sold as Arimidex® by AstraZeneca Pharmaceuticals LP; Wilmington, Del.),

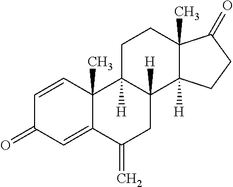

(exemestane; sold as Aromasin® by Pharmacia Corporation; Kalamazoo, Mich.) or

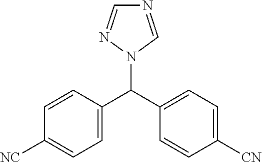

(letrozole; sold as Femara® by Novartis Pharmaceuticals Corporation; East Hanover, N.J.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with an estrogen such as DES(diethylstilbestrol),

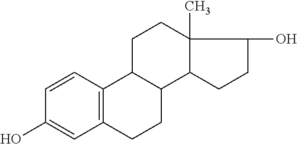

(estradiol; sold as Estrol® by Warner Chilcott, Inc.; Rockaway, N.J.) or conjugated estrogens (sold as Premarin® by Wyeth Pharmaceuticals Inc.; Philadelphia, Pa.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with anti-angiogenesis agents including bevacizumab (Avastin™; Genentech; San Francisco, Calif.), the anti-VEGFR-2 antibody IMC-1C11, other VEGFR inhibitors such as: CHIR-258

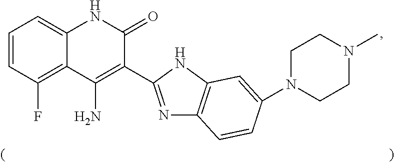

any of the inhibitors set forth in WO2004/13145 (e.g., comprising the core structural formula:

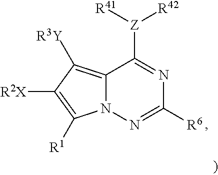

WO2004/09542 (e.g., comprising the core structural formula:

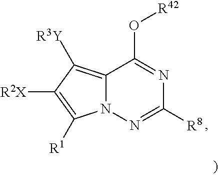

WO00/71129 (e.g., comprising the core structural formula:

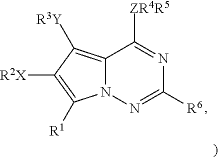

WO2004/09601 (e.g., comprising the core structural formula:

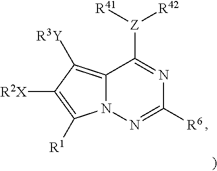

WO2004/01059 (e.g., comprising the core structural formula:

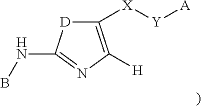

WO01/29025 (e.g., comprising the core structural formula:

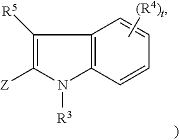

WO02/32861 (e.g., comprising the core structural formula:

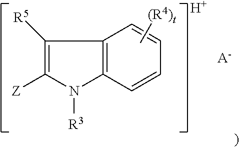

or set forth in WO03/88900 (e.g., comprising the core structural formula

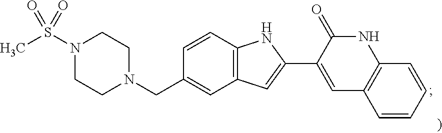

3-[5-(methylsulfonylpiperadinemethyl)-indolyl]-quinolone; Vatalanib

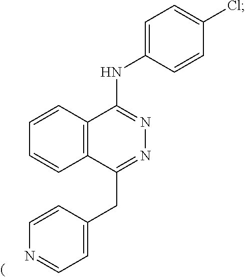

### PTK/ZK; CPG-79787; ZK-222584), AG-013736

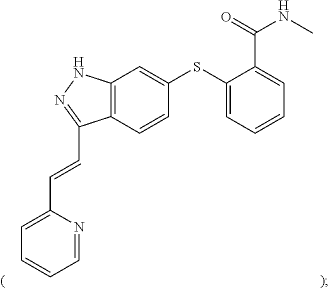

and the VEGF trap (AVE-0005), a soluble decoy receptor comprising portions of VEGF receptors 1 and 2. Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with a LHRH (Lutenizing hormone-releasing hormone) agonist such as the acetate salt of [D-Ser(But) 6, Azgly 10] (pyro-Glu-His-Trp-Ser-Tyr-D-Ser(But)-Leu-Arg-Pro-Azgly-NH₂ acetate [C₅₉H₈₄N₁₈O₁₄.(C₂H₄O₂)ₓ where x=1 to 2.4];

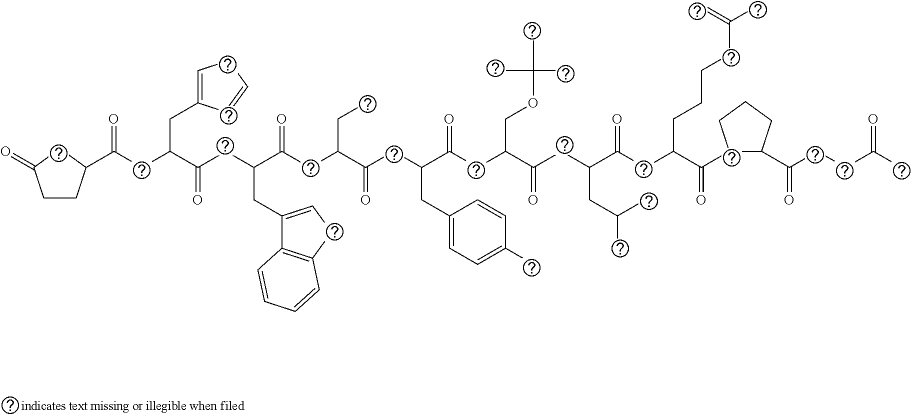

(goserelin acetate; sold as Zoladex® by AstraZeneca UK Limited; Macclesfield, England),

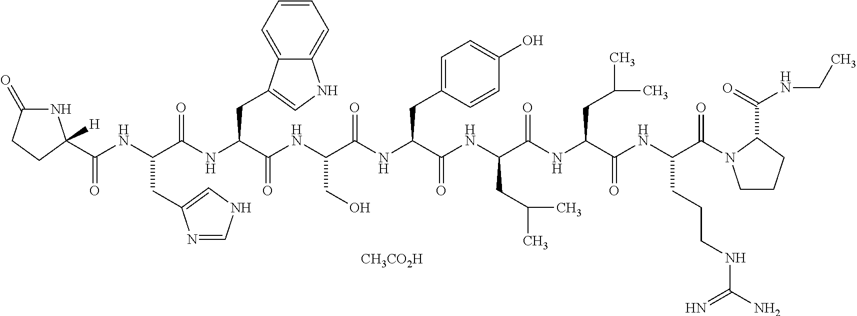

(leuprolide acetate; sold as Eligard® by Sanofi-Synthelabo Inc.; New York, N.Y.) or

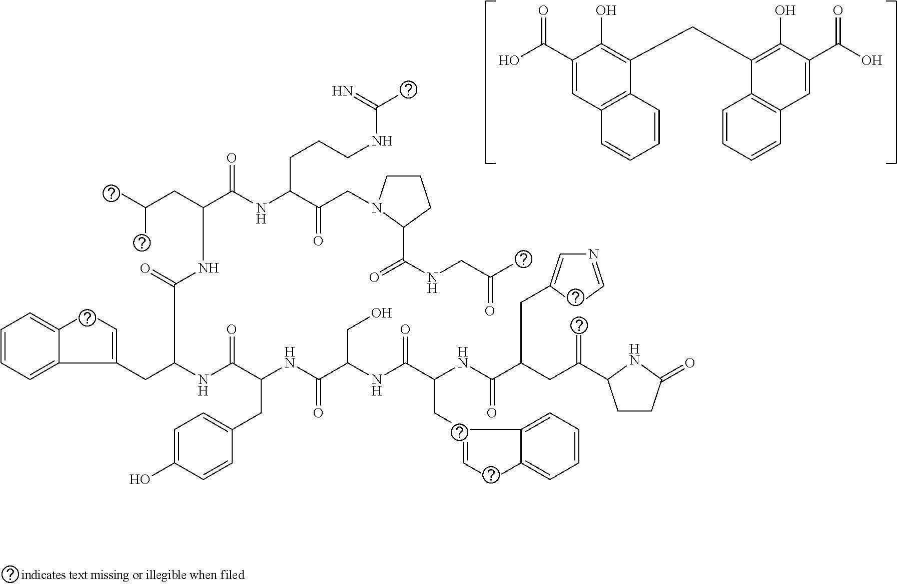

(triptorelin pamoate; sold as Trelstar® by Pharmacia Company, Kalamazoo, Mich.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with a progestational agent such as

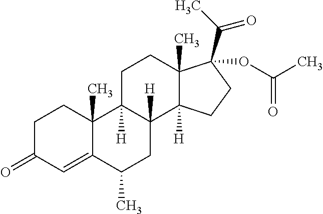

(medroxyprogesterone acetate; sold as Provera® by Pharmacia & Upjohn Co.; Kalamazoo, Mich.),

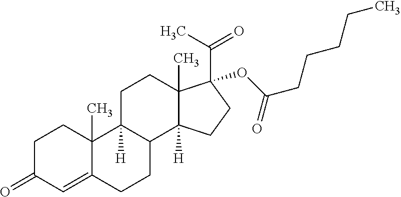

(hydroxyprogesterone caproate; 17-((1-Oxohexyl)oxy)pregn-4-ene-3,20-dione;), megestrol acetate or progestins. Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with selective estrogen receptor modulator (SERM) such as

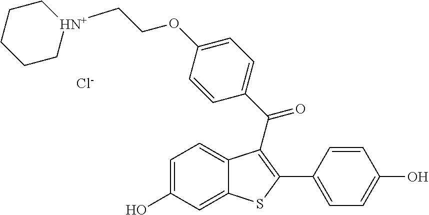

(raloxifene; sold as Evista® by Eli Lilly and Company; Indianapolis, Ind.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with an anti-androgen including, but not limited to:

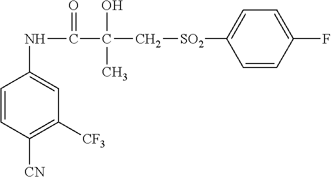

(bicalutamide; sold at CASODEX® by AstraZeneca Pharmaceuticals LP; Wilmington, Del.);

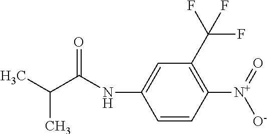

(flutamide; 2-methyl-N-[4-nitro-3 (trifluoromethyl) phenyl] propanamide; sold as Eulexin® by Schering Corporation; Kenilworth, N.J.);

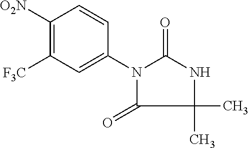

(nilutamide; sold as Nilandron® by Aventis Pharmaceuticals Inc.; Kansas City, Mo.) and

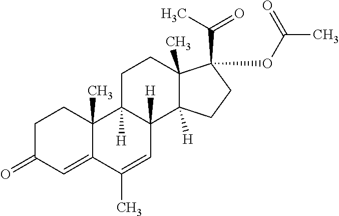

(Megestrol acetate; sold as Megace® by Bristol-Myers Squibb). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with one or more inhibitors which antagonize the action of the EGF Receptor or HER2, including, but not limited to, CP-724714

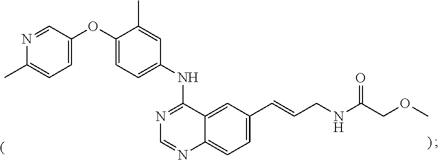

### TAK-165

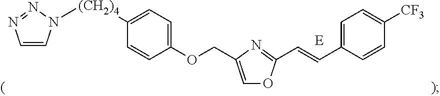

### HKI-272

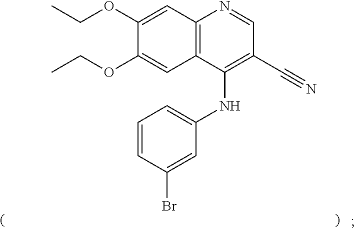

### OSI-774

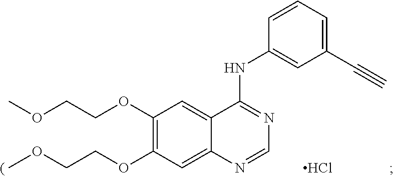

erlotinib, Hidalgo et al., J. Clin. Oncol. 19 (13): 3267-3279 (2001)), Lapatanib

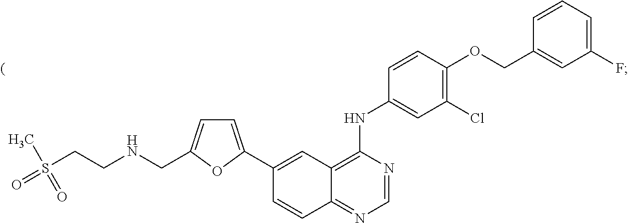

GW2016; Rusnak et al., Molecular Cancer Therapeutics 1:85-94 (2001); N-{3-Chloro-4-[(3-fluorobenzyl)oxy]phenyl}-6-[5-({[2-(methylsulfonyl)ethyl]amino}methyl)-2-furyl]-4-quinazolinamine; PCT Application No. WO99/35146), Canertinib (CI-1033;

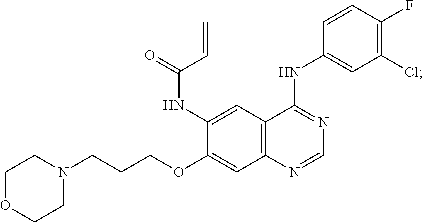

Erlichman et al., Cancer Res. 61 (2):739-48 (2001); Smaill et al., J. Med. Chem. 43 (7):1380-97 (2000)), ABX-EGF antibody (Abgenix, Inc.; Freemont, Calif.; Yang et al., Cancer Res. 59 (6):1236-43 (1999); Yang et al., Crit Rev Oncol Hematol. 38 (1):17-23 (2001)), erbitux (U.S. Pat. No. 6,217,866; IMC-C225, cetuximab; Imclone; New York, N.Y.), EKB-569

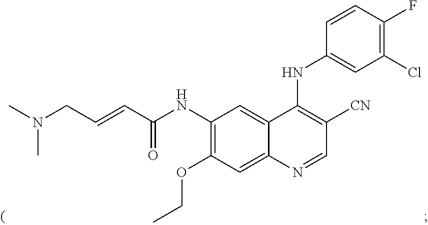

Wissner et al., J. Med. Chem. 46 (1): 49-63 (2003)), PKI-166

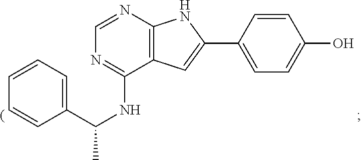

CGP-75166), GW-572016, any anti-EGFR antibody and any anti-HER2 antibody. Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with:

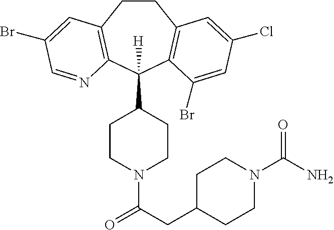

(lonafarnib; Sarasar™; Schering-Plough; Kenilworth, N.J.). In another embodiment, one of the following FPT inhibitors is provided in association with an IGF1R inhibitor:

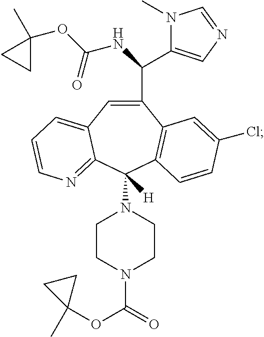

or

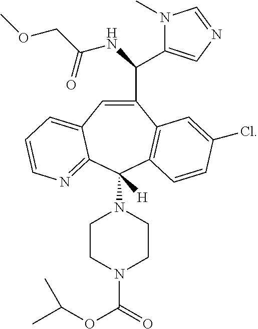

Methods of treating or preventing rhabdomyosarcoma, Wilm's tumor, osteosarcoma, neuroblastoma, pancreatic cancer or any pediatric cancer by administering these agents are within the scope of the present invention.

Other FPT inhibitors, that can be provided in association with an IGF1R inhibitor include BMS-214662

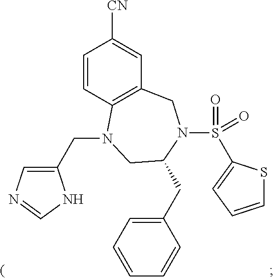

Hunt et al., J. Med. Chem. 43 (20):3587-95 (2000); Dancey et al., Curr. Pharm. Des. 8:2259-2267 (2002); (R)-7-cyano-2,3,4,5-tetrahydro-1-(1H-imidazol-4-ylmethyl)-3-(phenylmethyl)-4-(2-thienylsulfonyl)-1H-1,4-benzodiazepine)) and R155777 (tipifarnib; Garner et al., Drug Metab. Dispos. 30 (7):823-30 (2002); Dancey et al., Curr. Pharm. Des. 8:2259-2267 (2002); (B)-6-[amino(4-chlorophenyl)(1-methyl-1H-imidazol-5-yl)-methyl]-4-(3-chlorophenyl)-1-methyl-2(1H)-quinolinone];

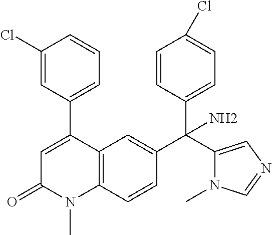

sold as Zarnestra™; Johnson & Johnson; New Brunswick, N.J.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with

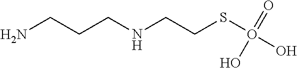

### (Amifostine);

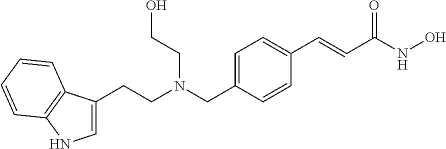

### (NVP-LAQ824; Atadja et al., Cancer Research 64: 689-695 (2004)),

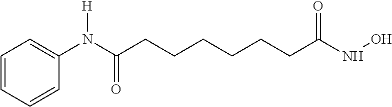

(suberoyl analide hydroxamic acid),

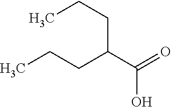

(Valproic acid; Michaelis et al., Mol. Pharmacol. 65:520-527 (2004)),

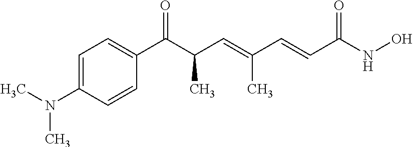

(trichostatin A),

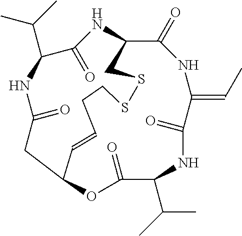

### (FK-228; Furumai et al., Cancer Research 62: 4916-4921 (2002)),

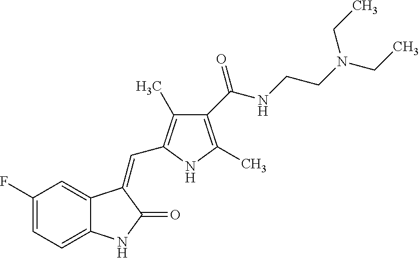

(SU11248; Mendel et al., Clin. Cancer Res. 9 (1):327-37 (2003)),

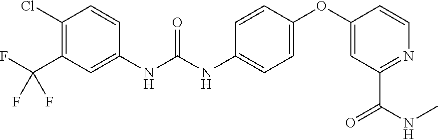

### (BAY43-9006),

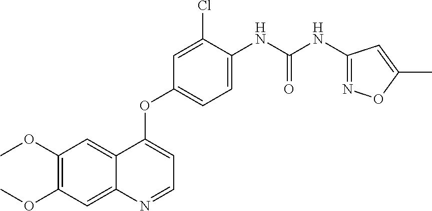

### (KRN951),

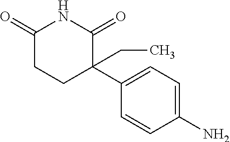

### (Aminoglutethimide);

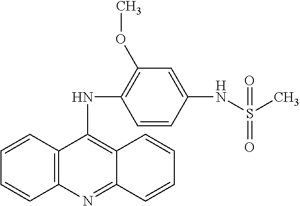

### (Amsacrine);

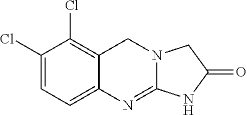

### (Anagrelide);

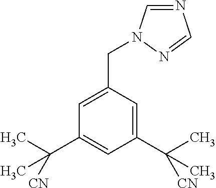

(Anastrozole; sold as Arimidex by AstraZeneca Pharmaceuticals LP; Wilmington, Del.); Asparaginase; Bacillus Calmette-Guerin (BCG) vaccine (Garrido et al., Cytobios. 90 (360):47-65 (1997));

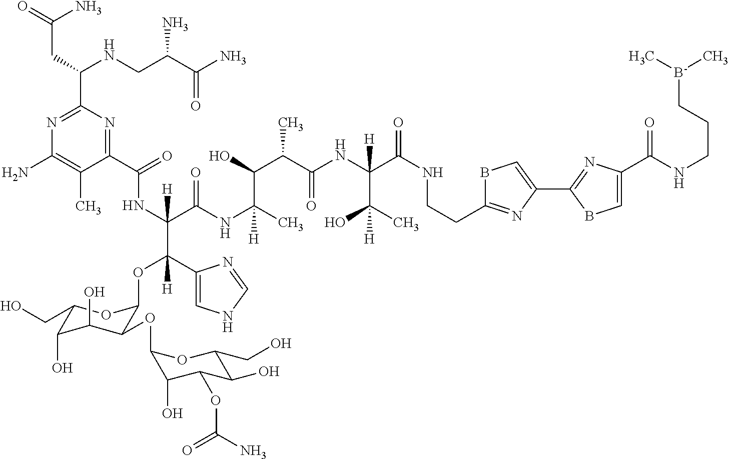

### (Bleomycin);

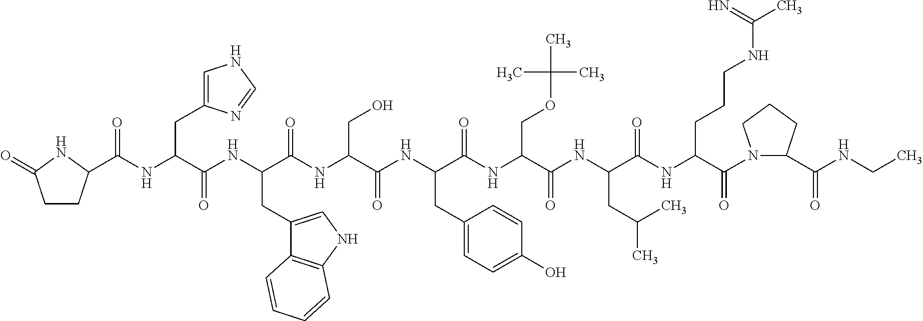

### (Buserelin);

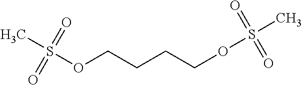

(Busulfan; 1,4-butanediol, dimethanesulfonate; sold as Busulfex® by ESP Pharma, Inc.; Edison, N.J.);

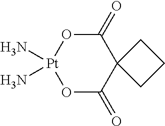

(Carboplatin; sold as Paraplatin® by Bristol-Myers Squibb; Princeton, N.J.);

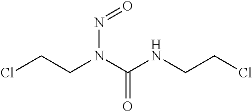

### (Carmustine);

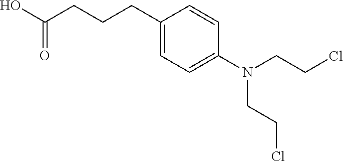

### (Chlorambucil);

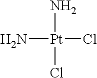

### (Cisplatin);

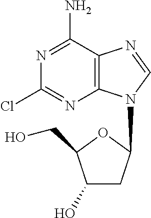

### (Cladribine);

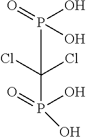

### (Clodronate);

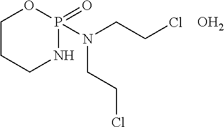

### (Cyclophosphamide);

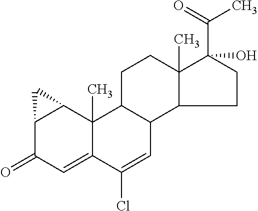

### (Cyproterone);

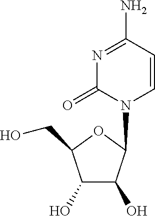

### (Cytarabine);

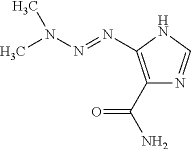

### (Dacarbazine);

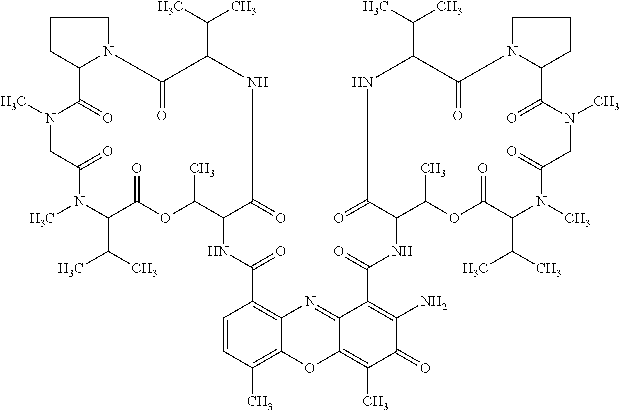

### (Dactinomycin);

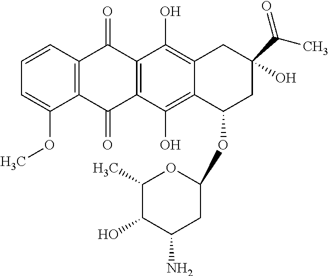

### (Daunorubicin);

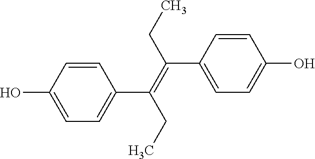

### (Diethylstilbestrol);

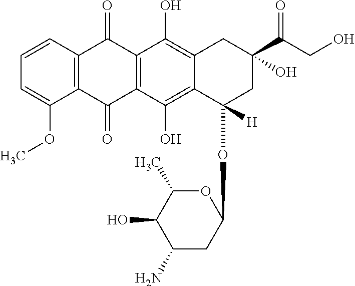

### (Epirubicin);

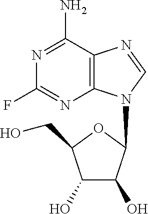

### (Fludarabine);

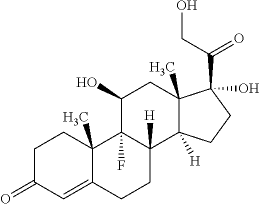

### (Fludrocortisone);

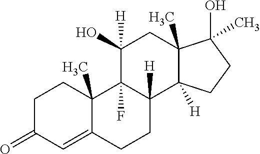

### (Fluoxymesterone);

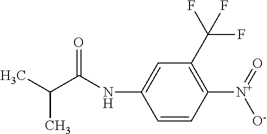

### (Flutamide);

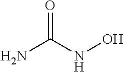

### (Hydroxyurea);

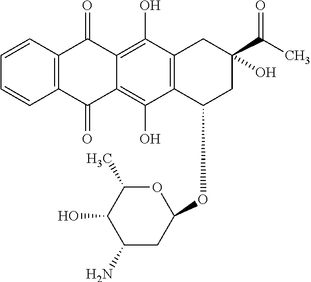

### (Idarubicin);

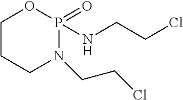

### (Ifosfamide)

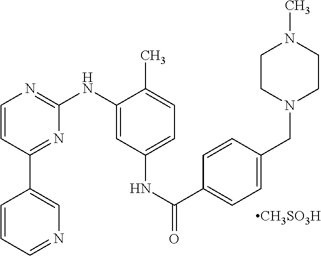

(Imatinib; sold as Gleevec® by Novartis Pharmaceuticals Corporation; East Hanover, N.J.);

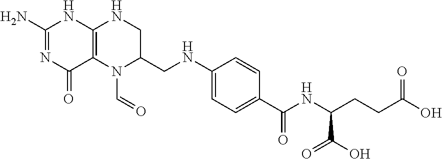

### (Leucovorin);

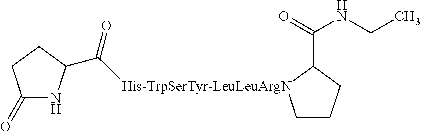

### (Leuprolide);

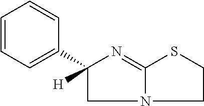

### (Levamisole);

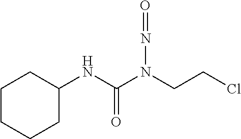

### (Lomustine);

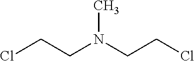

### (Mechlorethamine);

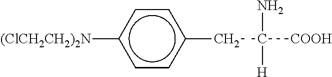

(Melphalan; sold as Alkeran® by Celgene Corporation; Warren, N.J.);

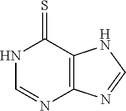

### (Mercaptopurine);

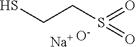

### (Mesna);

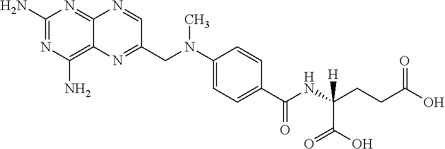

### (Methotrexate);

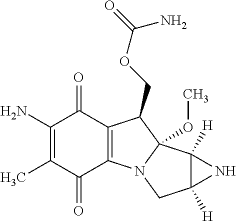

### (Mitomycin);

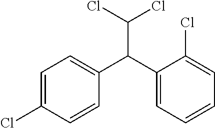

### (Mitotane);

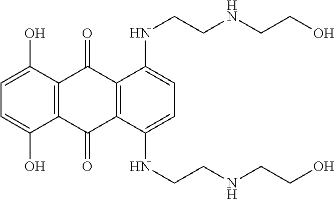

### (Mitoxantrone);

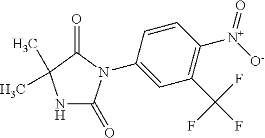

(Nilutamide); octreotide (L-Cysteinamide, D-phenylalanyl-L-cysteinyl-L-phenylalanyl-D-tryptophyl-L-lysyl-L-threonyl-N-[2-hydroxy-1-(hydroxymethyl) propyl]-, cyclic (2_{—}7)-disulfide; [R R*,R*)];

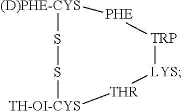

Katz et al., Clin Pharm. 8(4):255-73 (1989); sold as Sandostatin LAR® Depot; Novartis Pharm. Corp; E. Hanover, N.J.); oxaliplatin (

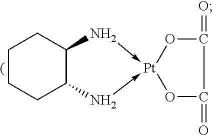

sold as Eloxatin™ by Sanofi-Synthelabo Inc.; New York, N.Y.);

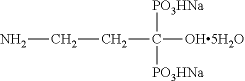

(Pamidronate; sold as Aredia® by Novartis Pharmaceuticals Corporation; East Hanover, N.J.);

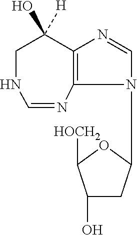

(Pentostatin; sold as Nipent® by Supergen; Dublin, Calif.);

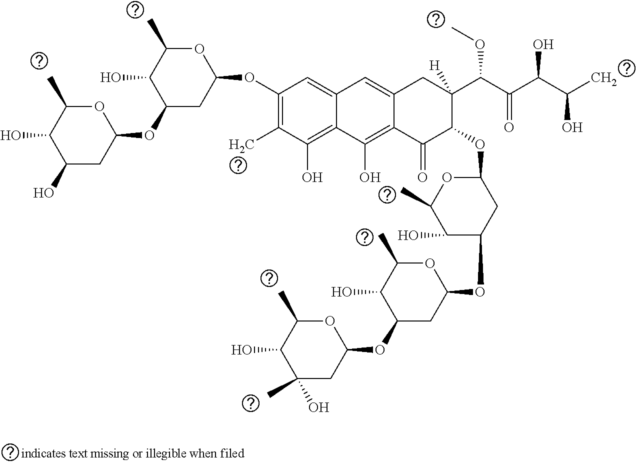

### (Plicamycin);

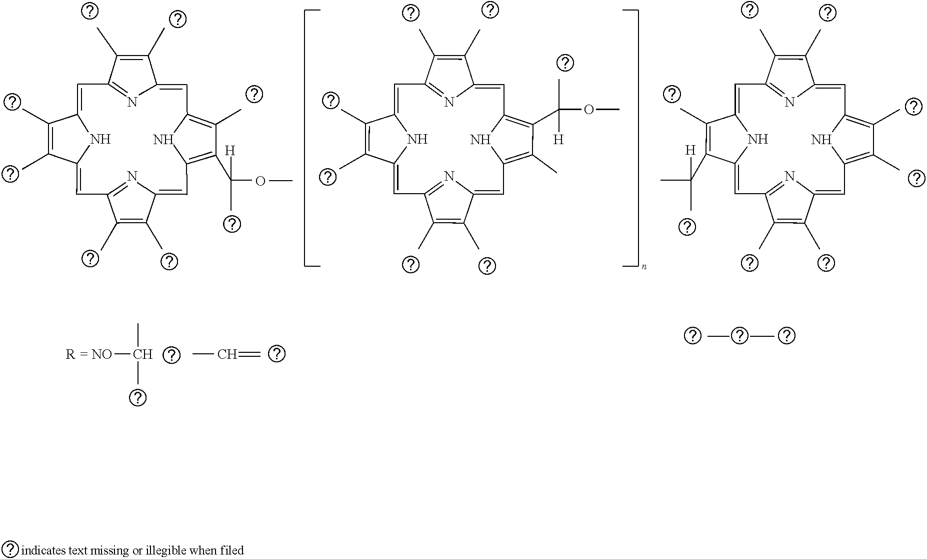

(Porfimer; sold as Photofrin® by Axcan Scandipharm Inc.; Birmingham, Ala.);

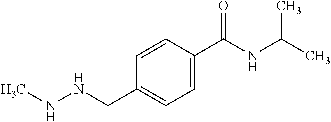

### (Procarbazine);

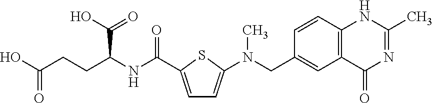

(Raltitrexed); Rituximab (sold as Rituxan® by Genentech, Inc.; South San Francisco, Calif.);

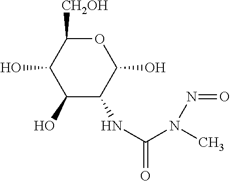

### (Streptozocin);

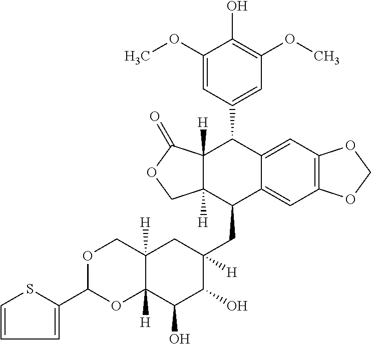

### (Teniposide);

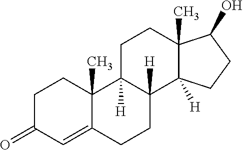

### (Testosterone);

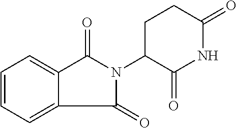

### (Thalidomide);

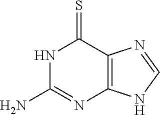

### (Thioguanine);

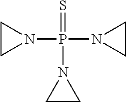

### (Thiotepa);

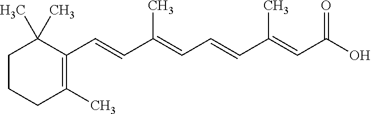

### (Tretinoin);

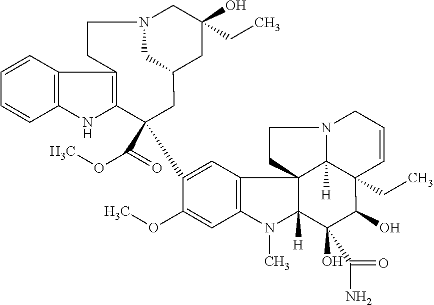

(Vindesine) or 13-cis-retinoic acid

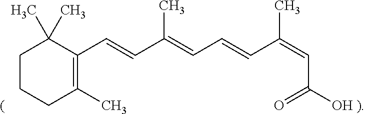

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with one or more of any of: phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12,1M862, angiostatin, vitaxin, droloxifene, idoxyfene, spironolactone, finasteride, cimitidine, trastuzumab, denileukin, diftitox, gefitinib, bortezimib, paclitaxel, docetaxel, epithilone B, BMS-247550 (see e.g., Lee et al., Clin. Cancer Res. 7:1429-1437 (2001)), BMS-310705, droloxifene (3-hydroxytamoxifen), 4-hydroxytamoxifen, pipendoxifene, ERA-923, arzoxifene, fulvestrant, acolbifene, lasofoxifene (CP-336156), idoxifene, TSE-424, HMR-3339, ZK186619, topotecan, PTK787/ZK 222584 (Thomas et al., Semin Oncol. 30 (3 Suppl 6):32-8 (2003)), the humanized anti-VEGF antibody Bevacizumab, VX-745 (Haddad, Curr Opin. Investig. Drugs 2 (8):1070-6 (2001)), PD 184352 (Sebolt-Leopold, et al. Nature Med. 5: 810-816 (1999)), rapamycin, CCI-779 (Sehgal et al., Med. Res. Rev., 14:1-22 (1994); Elit, Curr. Opin. Investig. Drugs 3 (8):1249-53 (2002)), LY294002, LY292223, LY292696, LY293684, LY293646 (Vlahos et al., J. Biol. Chem. 269 (7): 5241-5248 (1994)), wortmannin, BAY-43-9006, (Wilhelm et al., Curr. Pharm. Des. 8:2255-2257 (2002)), ZM336372, L-779,450, any Raf inhibitor disclosed in Lowinger et al., Curr. Pharm Des. 8:2269-2278 (2002); flavopiridol (L86-8275/HMR 1275; Senderowicz, Oncogene 19 (56): 6600-6606 (2000)) or UCN-01 (7-hydroxy staurosporine; Senderowicz, Oncogene 19 (56): 6600-6606 (2000)). Methods of treating or preventing rhabdomyosarcoma, Wilm's tumor, osteosarcoma, neuroblastoma, pancreatic cancer or any pediatric cancer by administering these agents are within the scope of the present invention.

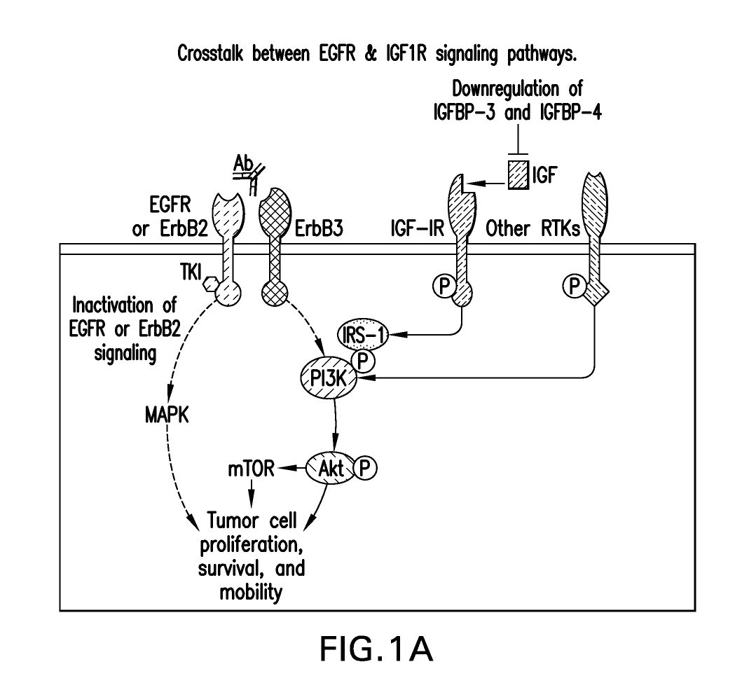

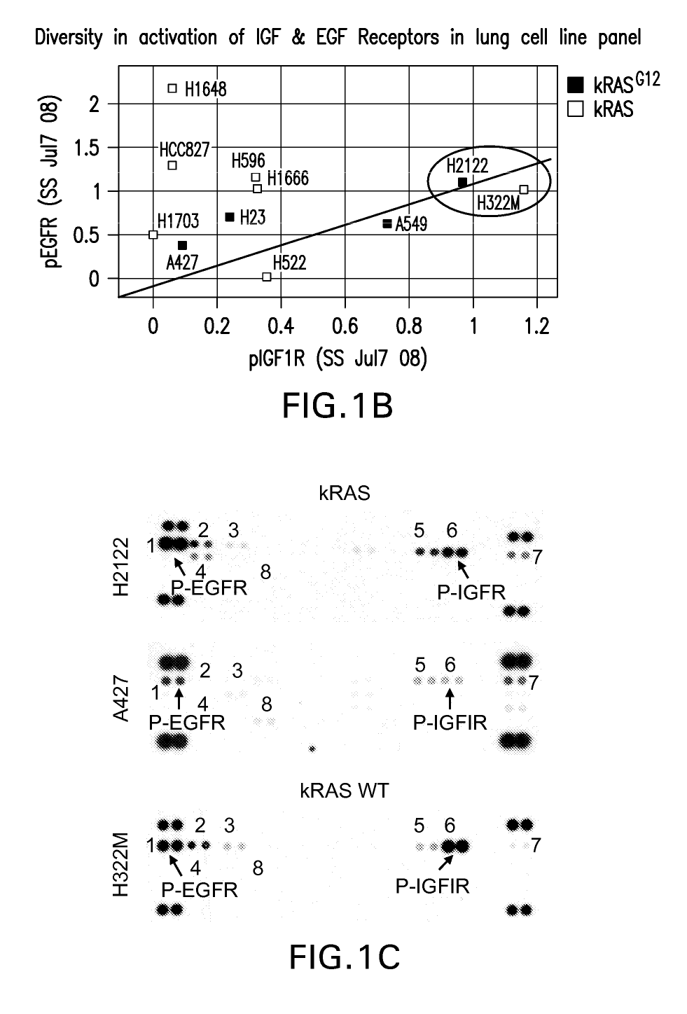

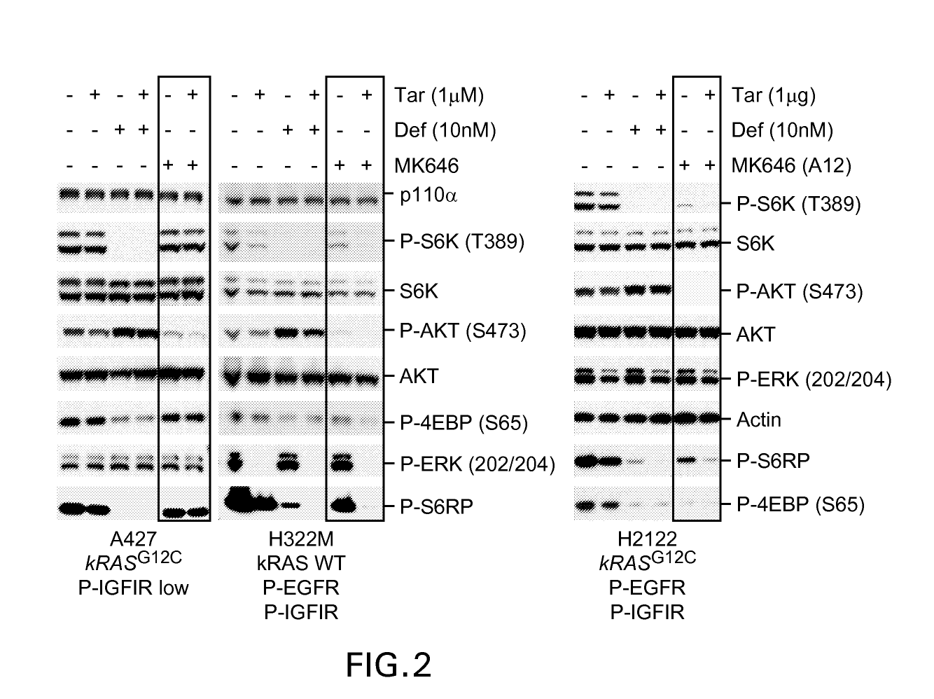

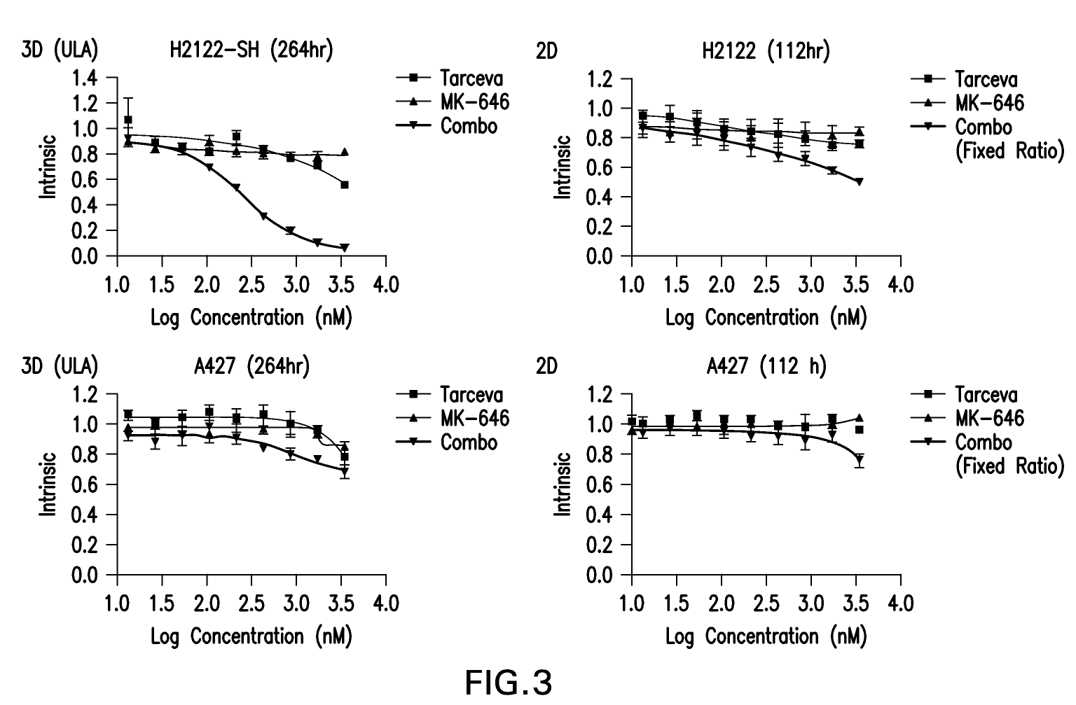

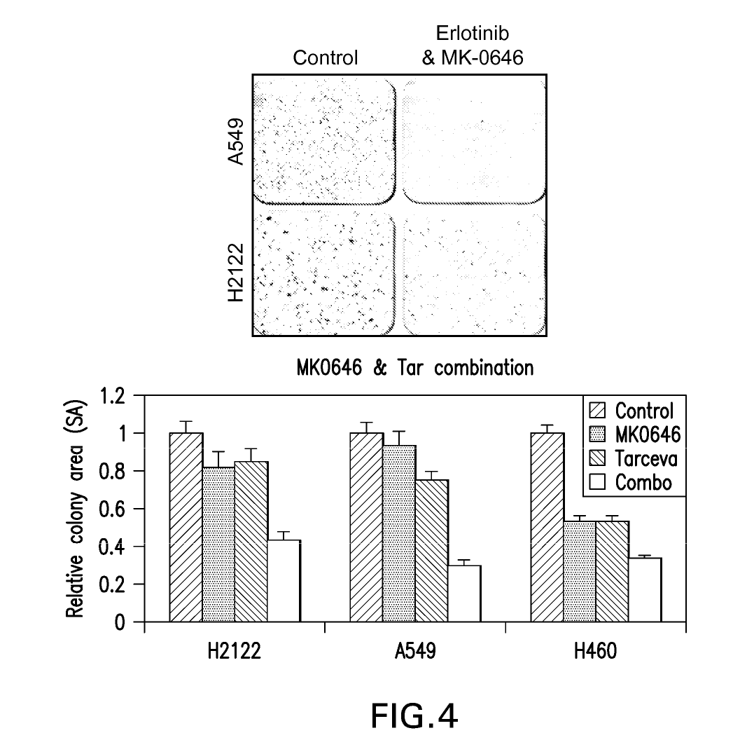

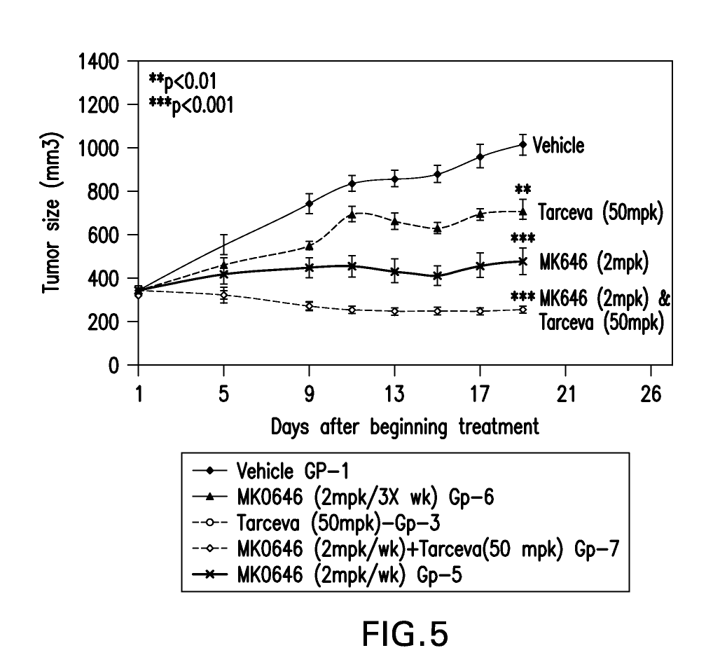

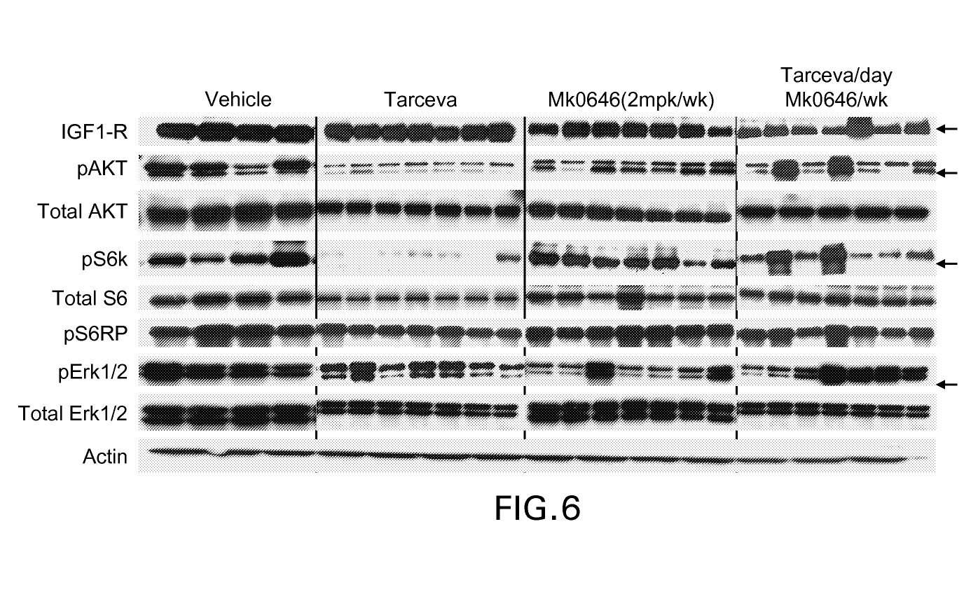

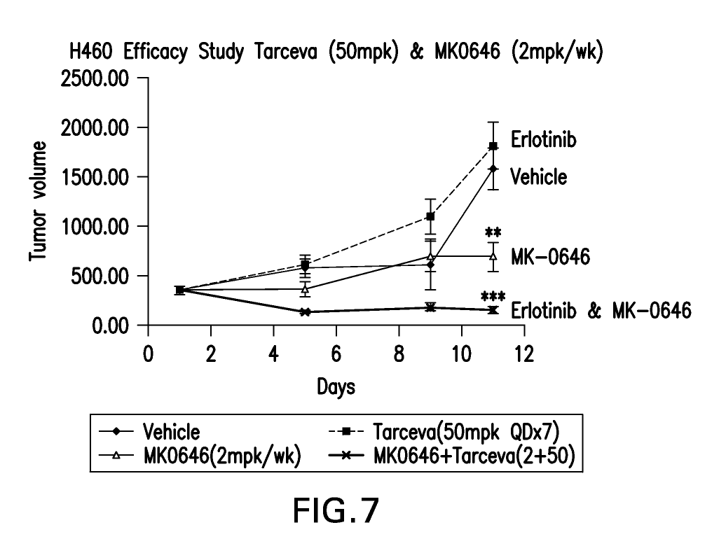

In an embodiment of the invention, an IGF1R inhibitor is provided in association with one or more of any of the compounds set forth in U.S. Pat. No. 5,656,655, which discloses styryl substituted heteroaryl EGFR inhibitors; in U.S. Pat. No. 5,646,153 which discloses bis mono and/or bicyclic aryl heteroaryl carbocyclic and heterocarbocyclic EGFR and PDGFR inhibitors; in U.S. Pat. No. 5,679,683 which discloses tricyclic pyrimidine compounds that inhibit the EGFR; in U.S. Pat. No. 5,616,582 which discloses quinazoline derivatives that have receptor tyrosine kinase inhibitory activity; in Fry et al., Science 265 1093-1095 (1994) which discloses a compound having a structure that inhibits EGFR (see FIG. 1 of Fry et al.); in U.S. Pat. No. 5,196,446 which discloses heteroarylethenediyl or heteroarylethenediylaryl compounds that inhibit EGFR; in Panek, et al., Journal of Pharmacology and Experimental Therapeutics 283: 1433-1444 (1997) which disclose a compound identified as PD166285 that inhibits the EGFR, PDGFR, and FGFR families of receptors-PD166285 is identified as 6-(2,6-dichlorophenyl)-2-(4-(2-diethylaminoethoxy)phenylamino)-8-methyl-8H-pyrido(2,3-d)pyrimidin-7-one. Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with one or more of any of: pegylated or unpegylated interferon alfa-2a, pegylated or unpegylated interferon alfa-2b, pegylated or unpegylated interferon alfa-2c, pegylated or unpegylated interferon alfa n-1, pegylated or unpegylated interferon alfa n-3 and pegylated, unpegylated consensus interferon or albumin-interferon-alpha. Methods of treating or preventing rhabdomyosarcoma, Wilm's tumor, osteosarcoma, neuroblastoma, pancreatic cancer or any pediatric cancer by administering these agents are within the scope of the present invention.

The term “interferon alpha” as used herein means the family of highly homologous species-specific proteins that inhibit cellular proliferation and modulate immune response. Typical suitable interferon-alphas include, but are not limited to, recombinant interferon alpha-2b, recombinant interferon alpha-2a, recombinant interferon alpha-2c, alpha 2 interferon, interferon alpha-n1 (INS), a purified blend of natural alpha interferons, a consensus alpha interferon such as those described in U.S. Pat. Nos. 4,897,471 and 4,695,623 (especially Examples 7, 8 or 9 thereof), or interferon alpha-n3, a mixture of natural alpha interferons.

Interferon alfa-2a is sold as ROFERON-A® by Hoffmann-La Roche (Nutley, N.J.).

Interferon alfa-2b is sold as INTRON-A® by Schering Corporation (Kenilworth, N.J.). The manufacture of interferon alpha 2b is described, for example, in U.S. Pat. No. 4,530,901.

Interferon alfa-n3 is a mixture of natural interferons sold as ALFERON N INJECTION® by Hemispherx Biopharma, Inc. (Philadelphia, Pa.).

Interferon alfa-n1 (INS) is a mixture of natural interferons sold as WELLFERON® by Glaxo-Smith-Kline (Research Triangle Park, N.C.).

Consensus interferon is sold as INFERGEN® by Intermune, Inc. (Brisbane, Calif.).

Interferon alfa-2c is sold as BEROFOR® by Boehringer Ingelheim Pharmaceutical, Inc. (Ridgefield, Conn.).

A purified blend of natural interferons is sold as SUMIFERON® by Sumitomo; Tokyo, Japan.

The term “pegylated interferon alpha” as used herein means polyethylene glycol modified conjugates of interferon alpha, preferably interferon alpha-2a and alpha-2b. The preferred polyethylene-glycol-interferon alpha-2b conjugate is PEG 12000-interferon alpha-2b. The phrases “12,000 molecular weight polyethylene glycol conjugated interferon alpha” and “PEG 12000-IFN alpha” as used herein include conjugates such as are prepared according to the methods of International Application No. WO 95/13090 and containing urethane linkages between the interferon alpha-2a or -2b amino groups and polyethylene glycol having an average molecular weight of 12000. The pegylated inteferon alpha, PEG 12000-IFN-alpha-2b is available from Schering-Plough Research Institute, Kenilworth, N.J.

The preferred PEG 12000-interferon alpha-2b can be prepared by attaching a PEG polymer to the epsilon amino group of a lysine residue in the interferon alpha-2b molecule. A single PEG 12000 molecule can be conjugated to free amino groups on an IFN alpha-2b molecule via a urethane linkage. This conjugate is characterized by the molecular weight of PEG 12000 attached. The PEG 12000-IFN alpha-2b conjugate can be formulated as a lyophilized powder for injection.

Pegylated interferon alfa-2b is sold as PEG-INTRON® by Schering Corporation (Kenilworth, N.J.).

Pegylated interferon-alfa-2a is sold as PEGASYS® by Hoffmann-La Roche (Nutley, N.J.).

Other interferon alpha conjugates can be prepared by coupling an interferon alpha to a water-soluble polymer. A non-limiting list of such polymers includes-other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described, for example, in U.S. Pat. No. 4,766,106, U.S. Pat. No. 4,917, 888, European Patent Application No. 0 236 987 or 0 593 868 or International Publication No. WO 95/13090.

Pharmaceutical compositions of pegylated interferon alpha suitable for parenteral administration can be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g., NaCl), preservatives (e.g., thimerosol, cresol or benzyl alcohol), and surfactants (e.g., tween or polysorbates) in sterile water for injection. The pegylated interferon alpha can be stored as lyophilized powder under refrigeration at 2°-8° C. The reconstituted aqueous solutions are stable when stored between 2° and 8° C. and used within 24 hours of reconstitution. See for example U.S. Pat. Nos. 4,492,537; 5,762,923 and 5,766,582. The reconstituted aqueous solutions may also be stored in prefilled, multi-dose syringes such as those useful for delivery of drugs such as insulin. Typical, suitable syringes include systems comprising a prefilled vial attached to a pen-type syringe such as the NOVOLET® Novo Pen available from Novo Nordisk or the REDIPEN®, available from Schering Corporation, Kenilworth, N.J. Other syringe systems include a pen-type syringe comprising a glass cartridge containing a diluent and lyophilized pegylated interferon alpha powder in a separate compartment.

The scope of the present invention also includes compositions comprising an IGF1R inhibitor in association with one or more other anti-cancer chemotherapeutic agents (e.g., as described herein) and optionally (i.e., with or without) in association with one or more antiemetics including, but not limited to, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co,; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Compositions comprising an antiemetic are useful for preventing or treating nausea; a common side effect of anti-cancer chemotherapy. Accordingly, the present invention also includes methods for treating or preventing cancer in a subject by administering an IGF1R inhibitor optionally in association with one or more other chemotherapeutic agents (e.g., as described herein) and optionally in association with one or more antiemetics.

The present invention further comprises a method for treating or preventing any stage or type of neuroblastoma, rhabdomyosarcoma, Wilm's tumor, osteosarcoma, pancreatic cancer or any pediatric cancer by administering an IGFR inhibitory agent in association with a therapeutic procedure such as surgical tumorectomy or anti-cancer radiation treatment; optionally in association with a further chemotherapeutic agent and/or antiemetic, for example, as set forth above.

### Erlotinib

A broad aspect of the invention provides methods of effectively treating cancers without significant adverse effects to the human patient subject to treatment. The clinical outcomes of the treatment according to the invention are somewhat unexpected, in that the combination therapeutic comprising an anti-IGF-1R antibody and erlotinib are thought to be more effective in treating erlotinib resistant cancers. As well, the combination therapeutic (combination of MK-0646 and Erlotinib) is thought to be more effective in treating various cancers than erlotinib by itself. It is understood that other tyrosine kinase inhibitor may be combined with the IGF-1R antibody. Alternatively, the combination therapeutic may comprise more than one tyrosine kinase inhibitor thus comprising an anti-IGF-1R antibody combined with a chemotherapy cocktail comprising at least two or more chemotherapeutic agents which do not significantly increase incident occurrences of adverse events, when compared with the chemotherapeutic alone.

Receptor tyrosine kinases are large enzymes which span the cell membrane and possess an extracellular binding domain for growth factors such as epidermal growth factor, a transmembrane domain, and an intracellular portion which functions as a kinase to phosphorylate specific tyrosine residues in proteins and hence to influence cell proliferation. It is known that such kinases are frequently aberrantly expressed in common human cancers such as lung carcinoma, breast cancer, gastrointestinal cancer such as colon, rectal or stomach cancer, leukemia, and ovarian, bronchial or pancreatic cancer. It has also been shown that epidermal growth factor receptor (EGFR) which possesses tyrosine kinase activity is mutated and/or overexpressed in many human cancers such as brain, lung, squamous cell, bladder, gastric, breast, head and neck, esophageal, gynecological and thyroid tumors.

Accordingly, it has been recognized that inhibitors of receptor tyrosine kinases are useful as a selective inhibitors of the growth of mammalian cancer cells. For example, erbstatin, a tyrosine kinase inhibitor selectively attenuates the growth in athymic nude mice of a transplanted human mammary carcinoma which expresses epidermal growth factor receptor tyrosine kinase (EGFR) but is without effect on the growth of another carcinoma which does not express the EGF receptor.

Various other compounds, such as styrene derivatives, have also been shown to possess tyrosine kinase inhibitory properties. More recently five European patent publications, namely EP 0 566 226 A1, EP 0 602 851 A1, EP 0 635 507 A1, EP 0 635 498 A1 and EP 0 520 722 A1 have disclosed that certain quinazoline derivatives possess anti-cancer properties which result from their tyrosine kinase inhibitory properties. Also PCT publication WO 92/20642 discloses bis-mono and bicyclic aryl and heteroaryl compounds as tyrosine kinase inhibitors. Methods of making and using erlotinib are described and claimed in U.S. Pat. No. 5,747,498, filed May 28, 1996, and currently assigned to Pfizer Inc., the entire content of which is incorporated by reference herein.

### Dose and Route of Administration

The combination therapeutic comprising IGF-1R specific antibodies and chemotherapeutic agents of the invention are administered to a human patient, in accord with known methods, such as intravenous administration as a bolus or by continuous infusion over a period of time, by intramuscular, intraperitoneal, intracerobrospinal, subcutaneous, intraarticular, intrasynovial, intrathecal, oral, topical, or inhalation routes. Intravenous or subcutaneous administration of the antibody is preferred. Three distinct delivery approaches are expected to be useful for delivery of the antibodies in accordance with the invention. Conventional intravenous delivery will presumably be the standard delivery technique for the majority of tumours. However, in connection with some tumours, such as those in the peritoneal cavity exemplified by tumours of the ovaries, biliary duct, other ducts, and the like, intraperitoneal administration may prove favorable for obtaining high dose of antibody at the tumour and to minimize antibody clearance. In a similar manner certain solid tumours possess vasculature that is appropriate for regional perfusion. Regional perfusion will allow the obtention of a high dose of the antibody at the site of a tumour and will minimize short term clearance of the antibody.

As with any protein or antibody infusion based therapeutic, safety concerns are related primarily to (i) cytokine release syndrome, i.e., hypotension, fever, shaking, chills, (ii) the development of an immunogenic response to the material (i.e., development of human antibodies by the patient to the antibody therapeutic, or HAHA or HACA response), and (iii) toxicity to normal cells that express the EGF receptor, e.g., hepatocytes which express EGFR and/or IGF-1R. Standard tests and follow up will be utilized to monitor each of these safety concerns. In particular, liver function will be monitored frequently during clinical trails in order to assess damage to the liver, if any.

For the prevention or treatment of disease, the appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. In a combination therapy regimen, the compositions of the present invention are administered in a therapeutically effective or synergistic amount. As used herein, a therapeutically effective amount is such that co-administration of anti-IGF-1R antibody and one or more other therapeutic agents, or administration of a composition of the present invention, results in reduction or inhibition of the targeting disease or condition. A therapeutically synergistic amount is that amount of anti-IGF-1R antibody and one or more other therapeutic agents necessary to synergistically or significantly reduce or eliminate conditions or symptoms associated with a particular disease.

In a broad embodiment, the treatment of the present invention involves the combined administration of an anti-IGF-1R antibody and one or more chemotherapeutic agents. The combined administration includes co administration, using separate formulations or a single pharmaceutical formulation, and consecutive administration in either order, wherein preferably there is a time period while both (or all) active agents simultaneously exert their biological activities. Preparation and dosing schedules for such chemotherapeutic agents may be used according to manufacturers' instructions or as determined empirically by the skilled practitioner. Preparation and dosing schedules for chemotherapy are also described in Chemotherapy Service Ed., M. C. Perry, Williams & Wilkins, Baltimore, Md. (1992). The chemotherapeutic agent may precede, or follow administration of the antibody or may be given simultaneously therewith. The clinical dosing of therapeutic combination of the present invention are likely to be limited by the extent of adverse reactions skin rash as observed with monoclonal anti-IGF-1R antibodies and a tyrosine kinase inhibitor (TKI) (Erlotinib and Gefitinib) used in the clinic today.

The term “therapeutically effective amount” or “therapeutically effective dosage” means that amount or dosage of a composition of the invention (e.g., IGF1R inhibitor, such as an anti-IGF1R antibody) that will elicit a biological or medical response of a tissue, system, subject or host that is being sought by the administrator (such as a researcher, doctor or veterinarian) which includes any measurable alleviation of the signs, symptoms and/or clinical indicia of cancer, such as non-small cell lung cancer or any other Erlotinib or IGF-1R resistant cancer (e.g., tumor growth) and/or the prevention, slowing or halting of progression or metastasis of the cancer to any degree.

Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved. Further information about suitable dosages is provided in the Example below.

For example, in one embodiment, a “therapeutically effective dosage” of any anti-IGF1R antibody; for example, an antibody or antigen-binding fragment thereof corresponding to Dolutuzumab or any other anti-IGF1R antibody mentioned herein is between about 40 and about 1000 mg/m² (e.g., about 50 mg/m², 60 mg/m², 70 mg/m², 80 mg/m², 90 mg/m², 100 mg/m², about 200 mg/m², about 300 mg/m², about 400 mg/m², about 500 mg/m², about 600 mg/m² or about 700 mg/m²) or 1-20 mg/kg of body weight (e.g., about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, about 20 mg/kg of body weight), once per week.

Dosage regimens may be adjusted to provide the optimum desired response (e.g., a therapeutic response). For example, a single dose may be administered or several divided doses may be administered over time or the dose may be proportionally reduced or increased as indicated by exigencies of the therapeutic situation. For example, dosage may be determined or adjusted, by a practitioner of ordinary skill in the art (e.g., physician or veterinarian) according to the patient's age, weight, height, past medical history, present medications and the potential for cross-reaction, allergies, sensitivities and adverse side-effects. It is especially advantageous to formulate parenteral compositions in dosage unit form for ease of administration and uniformity of dosage.

A physician or veterinarian having ordinary skill in the art can readily determine and prescribe the effective amount of the pharmaceutical composition required. For example, the physician or veterinarian could start doses of the antibody or antigen-binding fragment of the invention employed in the pharmaceutical composition at levels lower than that required in order to achieve the desired therapeutic effect and gradually increase the dosage until the desired effect is achieved. The effectiveness of a given dose or treatment regimen of an antibody or combination of the invention can be determined, for example, by determining whether a tumor being treated in the subject shrinks or ceases to grow. The size of tumor can be easily determined, for example, by X-ray, magnetic resonance imaging (MRI) or visually in a surgical procedure. Tumor size and proliferation can also be measured by use of a thymidine PET scan (see e.g., Wells et al., Clin. Oncol. 8: 7-14 (1996)). Generally, the thymidine PET scan includes the injection of a radioactive tracer, such as [2-¹¹C]-thymidine, followed by a PET scan of the patient's body (Vander Borght et al., Gastroenterology 101: 794-799, 1991; Vander Borght et al., J. Radiat. Appl. Instrum. Part A, 42: 103-104 (1991)). Other tracers that can be used include [¹⁸F]-FDG (18-fluorodeoxyglucose), [¹²⁴I]IUdR (5-[124I]iodo-2′-deoxyuridine), [⁷⁶Br]BrdUrd (Bromodeoxyuridine), [¹⁸F]FLT (3′-deoxy-3′fluorothymidine) or [¹¹C]FMAU (2′-fluoro-5-methyl-1-β-D-arabinofuranosyluracil).

For example, NSCLC progress can be monitored, by the physician or veterinarian by a variety of methods, and the dosing regimen can be altered accordingly. Methods by which to monitor progress include, for example, CT scan (e.g., to monitor tumor size), MRI scan (e.g., to monitor tumor size), chest X-ray (e.g., to monitor tumor size), bone scan, bone marrow biopsy, hormone tests, complete blood test (CBC), testing for NSCLC tumor markers in the urine or blood.

Depending on the type and severity of the disease, about 1 .μ.g/kg to 50 mg/kg (e.g. 0.1-20 mg/kg) of antibody is an initial candidate dosage for administration to the patient, whether, for example, by one or more separate administrations, or by continuous infusion. A typical daily dosage might range from about 1 .μ.g/kg to about 100 mg/kg or more, depending on the factors mentioned above. For repeated administrations over several days or longer, depending on the condition, the treatment is sustained until a desired suppression of disease symptoms occurs. However, other dosage regimens may be useful.

In one aspect, the antibody of the invention is administered weekly or may be administered every two to three weeks, at a dose ranged from about 5 mg/kg to about 15 mg/kg. More preferably, such dosing regimen is used in combination with a chemotherapy regimen for treating erlotinib resistant cancers such as NSCLC. In some aspects, the chemotherapy regimen involves the traditional high-dose intermittent administration. In some other aspects, the chemotherapeutic agents are administered using smaller and more frequent doses without scheduled breaks (“metronomic chemotherapy”). The progress of the therapy of the invention is easily monitored by conventional techniques and assays.

In one embodiment, the dosing sequence comprises administering erlotinib (oral) concurrently with the IGF-1R antibody—erlotinib is administered everyday while the IGF-1R antibody (MK-0646) is administered weekly. In particular, MK-0646 (IGF-1R mAb) is administered at a dose of 10 mg/kg i.v weekly while erlotinib is administered at 150 mg on a daily schedule.

Alternative dosing regiment for the IGF-1R antibody is as follows:

(i) 15 mg/kg loading, followed by 7.5 mg/kg every week.

(ii) 20 mg/kg every other week

(iii) 30 mg/kg every three weeks

For parenteral administration, the antibody can be formulated as a solution, suspension, emulsion or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by known or suitable techniques. The administration of the combination therapeutic may continue until disease progression.

While having described the invention in general terms, the embodiments of the invention will be further disclosed in the following examples.

### Articles of Manufacture

In another embodiment of the invention, an article of manufacture containing materials useful for the treatment of the disorders described above is provided. The article of manufacture comprises a container, a label and a package insert. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is an anti-IGF-1R antibody. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, and syringes. In addition, the article of manufacture comprises a package inserts with instructions for use, including for example instructing the user of the composition to administer the anti-IGF-1R antibody composition and an EGFR-inhibitor e.g., erlotinib composition to a patient.

All publications mentioned herein are incorporated herein by reference for the purpose of describing and disclosing, for example, the constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### EXAMPLE 1

### Correlation Between Activation of EGFR and IGF1R and Efficacy to MK-0646/Erlotinib Combination

Summary: Multiple receptor tyrosine kinase activation in a cell could contribute to drug resistance to Erlotinib. In fact activation of EGFR and cMET has been observed in clinical samples from Erlotinib resistant patients.

Methods: In order to identify tumors that would respond to MK-0646/Erlotinib combination, the phoshorylation status of various RTK in a panel of lung cancer cell lines was evaluated. The levels of activated EGFR and IGF1R across a panel of 10 lung cancer cell lines were quantified as shown in FIG. 1. Few cell lines represented by NCI-H2122 & NCI-H322M showed high levels of both P-IGFIR and P-EGFR, while the EGFR mutant cell line, HCC827 showed high levels of P-EGFR with little or no activation of IGFIR.

Briefly, all NSCLC cell lines were obtained from ATCC and maintained in DMEM or RPMI with 10% FBS as specified by ATCC. About 2 million cells were cultured in 10 cm plates and protein lysates were prepared from a sub-confluent culture and blotted on to a P-RTK array (R&D bioscience) as described by the manufacturer. The arrays were probed with HRP-conjugated P-Tyr antibody and then incubated with SuperSignal chemiluminescence substrate (Pierce) and blots were then exposed to a Kodak Biomax Light Film. The films were scanned and positions of the appropriate RTK spots (in duplicates) were aligned and intensities were determined using densitometry and quantified (Alpha Ease). Relative levels of P-RTKs were estimated by normalizing with the positive controls (P-Tyr peptides) spotted on the membrane (duplicate spots on four corners of the membrane).

### EXAMPLE 2

### Inhibition of P13K and RAS-MAPK Signaling by MK-0646/Erlotinib Combination

Summary: In order to test the effect of inhibition of these RTKs on P13K and RAS-MAPK pathway activity, the phosphorylation status of key nodes in the pathway were meaused. As shown in FIG. 2, combined inhibition of EGFR and IGFIR was more effective in blocking PI3K pathway as measured by the substantial decrease in P-S6RP & P-S6K in NCI-H-12122 & NCI-H322M cell lines that express high levels of both receptors. Such a synergistic inhibition of PI3K signaling could not be observed in cell lines with either low levels of both P-EGFR & P-IGFIR (A427 is shown as example). Similar results were obtained in other cell lines (data not shown).

Methods: For western blot analysis total protein lysates from cells (˜0.3 million) cultured in 6 well plates and treated with either Deforolimus (10 nM) or MK-0646 (10 ng/ml) or in combination for 4 hrs and harvested in SDS gel loading dye (Invitrogen). Samples were western blotted with indicated total or phosphospecific antibodies followed by a secondary antibody (Cell Signaling Technology, CST) and then incubated with SuperSignal chemiluminescence substrate (Pierce). The blots were then exposed to a Kodak Biomax Light Film. The antibodies against ERK, p-ERK (Thr202/Tyr204), AKT and p-AKT (Ser473), IGF1RI S6K & P-S6K (T389), IRS1 & P-IRS1 (S302) and actin were obtained from CST.

### EXAMPLE 3

### Functional Effect of Inhibiting Both EGFR and IGF-1 Signaling

Summary: To test the functional effect of inhibiting both EGFR & IGFIR signaling, the growth inhibition under adherent (2D) and non adherent (3D) conditions were evaluated. Under adherent growth conditions no significant growth inhibition was observed in MK-0646 treated cell lines. This is in agreement with prior experiments (data not shown). In order to test the effect of this combination under 3D non-adherent conditions, the inventors developed an ultra low attachment plate based proliferation assay. When grown under non-adherent conditions only 7/10 lines measurably grew and were used for sensitivity assessments. NCI-H2122 cells showed a substantial increase in sensitivity to Erlotinib/MK-0646 combination under non-adherent conditions. On the other hand, A427 cells with low levels of P-IGFIR and EGFR showed no significant growth inhibition under 2D or 3D growth conditions.

Methods: Cells (˜3×10̂3) were seeded in adherent or non-adherent (ultra-low attachment plates; Corning) 96 well plates. On day one cells were incubated with indicated concentrations of erlotinib or MK-0646 or the combination and a set of cells were harvested for DNA content measurements (Day 1). The media and drugs were slowly replaced every 3 days and at the end of the assay (as indicated) plates were harvested and DNA content was measured using Cyquant assays as described by the manufacturer. The intrinsic growth was calculated based on the increase in DNA content from Day 1 to the end of the assay.

### EXAMPLE 4

To confirm the above mentioned results, the inventors utilized a high-throughput soft agar colony formation assay. The anchorage independent growth was quantified using a fluorescent live cell dye (Lava Cell). The MK-0646 & Erlotinib combination significantly inhibited soft agar colony formation (FIG. 4: P>0.0001 compared to control) of both NCI-H2122 & A549 cell lines. H460 cells also showed increased growth inhibition in presence of the combination. Thus the in vitro analysis identified 3 out of 10 cell lines (30%) to respond better to the combination of MK-0646 & Erlotinib. This correlates with the activation of both RTKs (EGFR & IGF1R).

Methods: Soft agar assays were conducted in 96 well glass bottom plates (MatriCal). Cells were seeded at a concentration of 3,000-9,000 cells per well in 100 μl RMPI 1640 supplemented with 14% FBS and 0.3% (w/v) SeaPlaque Agarose (Lonza Rockland, Inc) on top of a bottom layer of consisting of the same culture media supplemented with 0.8% agarose. Compounds were added in 100 μl of culture media supplemented after agarose had solidified. Cells were incubated for 7-14 days before staining overnight with LavaCell (Active Motif). Colonies were quantified using an Isocyte™ laser scanning cytometer. The ability of MK-0646 to inhibit anchorage independent growth alone or in combination with standard of care agents was evaluated in a soft agar colony forming assay. The RTK status was evaluated in total protein lysates using the P-RTK arrays (R&D biosciences) as described by the manufacturer. The activating mutations in KRAS were identified from published cancer genome data bases (Sanger).

### EXAMPLE 5

### Evaluation of Erlotinib & MK-0646 Efficacy in a Kras Mutant Lung Tumor Xenograft Model

Previously, in vivo data from the k-RAS mutant NCI-H2122 xenograft (high p-IGF1R and p-EGF1R in vitro) showed good inhibition of tumor growth that correlated with IGF1R receptor down regulation. P13K pathway inhibition was also observed following MK-0646 treatment. Using this xenograft model, the inventors evaluated the effect of MK-0646 in combination with Erlotinib in Erlotinib resistant kRAS mutant patient population. As shown in FIG. 5, the combination of MK-0646 (2 mpk; once a week dosing) with Erlotinib (50 mpk) led to significant inhibition of tumor growth and even regression of the xenograft, thus providing further corroboration for the rationale underlying the combination of MK-0646 and Erlotinib for treating a pathology characterized by a kRAS mutant lung tumor.

Method: 2.5×106 NCI-H2122 human NSCLC cells were injected subcutaneously into the right flank of 4-6 week old nu/nu mice (Charles River Laboratories). When tumors reached a size of ˜300 mm3 (Length*Width*Width*0.5), mice were randomized into treatment groups. Mice (n=8/group) were dosed with vehicle once per week for 3 weeks (qwk×3) (20 mM L-Histidine, 150 mM NaCl, 0.5% PS80 pH=6) or 2 mpk of MK-0646 intra-peritoneal mg/kg MK-0646 qwk or Erlotinib (50 mg/kg by oral gavage) daily or in combination with MK-0646 for 3 weeks. Animals were weighed and tumor volumes were determined by calipering 2 times per week during the study and at termination. Tumor weight was determined at termination. On day 21 Animals were sacrificed by CO2 asphyxiation. Mice were sacrificed 24 hr after the final dose. At time of sacrifice, the tissue samples were collected and processed.

### EXAMPLE 6

### Correlation Between Efficacy and a Decrease in Total Protein Levels

Summary: As a measure of target engagement, total level of IGF1R was assayed via western blot ELISA (data not shown). The data showed a good correlation between efficacy and a decrease in total protein levels. As shown in FIG. 6, in each panel, MK-0646 is able to reduce the total levels of IGF1R when compared to vehicle or treatment with Erlotinib alone. These data suggest that total IGF1R levels may serve as a good biomarker for target engagement as well as efficacy over long term chronic treatments of MK-0646 alone or in combination with other targeted agents. Also a profound PI3K pathway inhibition as measured by decrease in P-AKT, PS6K & PS6RP was observed in the tumors treated with the combination. The RAS-MAPK pathway was also inhibited by the combination. These data suggest that combined inhibition of IGF1R and EGFR resulted in an increased inhibition of growth factor signaling resulting in anti-tumor efficacy relative to each agent being administered alone. Referring to FIG. 7, similar antitumor efficacy with erlotinib & MK-0646 combination was also observed in another KRAS mutant erlotinib refractory NSCLC model.

Methods: Total protein (500 microgram) from xenograft samples was isolated at the end of the efficacy study (4 weeks after indicated doses). Samples from 6 independent tumors were analyzed for east treatment. The proteins were western blotted and visualized as described previously (See FIG. 2).

SEQUENCE LISTING
SEQ. ID. NO. 1
Arg Ser Ser Gln Ser Ile Val His Ser Asn Gly Asn Thr Tyr Leu Gln -
SEQ. ID. NO. 2
Lys Val Ser Asn Arg Leu Tyr -
SEQ. ID. NO. 3
Phe Gln Gly Ser His Val Pro Trp Thr -
SEQ. ID. NO. 4
Gly Gly Tyr Leu Trp Asn -
SEQ. ID. NO. 5
Tyr Ile Ser Tyr Asp Gly Thr Asn Asn Tyr Lys Pro Ser Leu Lys Asp -
SEQ. ID. NO. 6
Tyr Gly Arg Val Phe Phe Asp Tyr -
SEQ. ID. NO. 7
Asp Val Val Met Thr Gln Ser Pro Leu Ser Leu Pro Val Thr Pro Gly Glu Pro Ala
Ser Ile Ser Cys Arg Ser Ser Gln Ser Ile Val His Ser Asn Gly Asn Thr Tyr Leu Gln Trp Tyr
Leu Gln Lys Pro Gly Gln Ser Pro Gln Leu Leu Ile Tyr Lys Val Ser Asn Arg Leu Tyr Gly Val
Pro Asp Arg Phe Ser Gly Ser Gly Ser Gly Thr Asp Phe Thr Leu Lys Ile Ser Arg Val Gln Ala
Glu Asp Val Gly Val Tyr Tyr Cys Phe Gln Gly Ser His Val Pro Trp Thr Phe Gly Gln Gly Thr
Lys Val Glu Ile Lys -
SEQ. ID. NO. 8
Asp Ile Val Met Thr Gln Ser Pro Leu Ser Leu Pro Val Thr Pro Gly Glu Pro Ala
Ser Ile Ser Cys Arg Ser Ser Gln Ser Ile Val His Ser Asn Gly Asn Thr Tyr Leu Gln Trp Tyr
Leu Gln Lys Pro Gly Gln Ser Pro Gln Leu Leu Ile Tyr Lys Val Ser Asn Arg Leu Tyr Gly Val
Pro Asp Arg Phe Ser Gly Ser Gly Ser Gly Thr Asp Phe Thr Leu Lys Ile Ser Arg Val Glu Ala
Glu Asp Val Gly Val Tyr Tyr Cys Phe Gln Gly Ser His Val Pro Trp Thr Phe Gly Gln Gly Thr
Lys Val Glu Ile Lys -
SEQ. ID. NO. 9
Gln Val Gln Leu Gln Glu Ser Gly Pro Gly Leu Val Lys Pro Ser Glu Thr Leu Ser
Leu Thr Cys Thr Val Ser Gly Tyr Ser Ile Thr Gly Gly Tyr Leu Trp Asn Trp Ile Arg Gln Pro
Pro Gly Lys Gly Leu Glu Trp Met Gly Tyr Ile Ser Tyr Asp Gly Thr Asn Asn Tyr Lys Pro Ser
Leu Lys Asp Arg Ile Thr Ile Ser Arg Asp Thr Ser Lys Asn Gln Phe Ser Leu Lys Leu Ser Ser
Val Thr Ala Ala Asp Thr Ala Val Tyr Tyr Cys Ala Arg Tyr Gly Arg Val Phe Phe Asp Tyr Trp
Gly Gln Gly Thr Leu Val Thr Val Ser Ser -
SEQ. ID. NO. 10
Gln Val Gln Leu Gln Glu Ser Gly Pro Gly Leu Val Lys Pro Ser Glu Thr Leu Ser
Leu Thr Cys Thr Val Ser Gly Tyr Ser Ile Thr Gly Gly Tyr Leu Trp Asn Trp Ile Arg Glu Pro
Pro Gly Lys Gly Leu Glu Trp Ile Gly Tyr Ile Ser Tyr Asp Gly Thr Asn Asn Tyr Lys Pro Ser
Leu Lys Asp Arg Val Thr Ile Ser Arg Asp Thr Ser Lys Asn Gln Phe Ser Leu Lys Leu Ser Ser
Val Thr Ala Ala Asp Thr Ala Val Tyr Tyr Cys Ala Arg Tyr Gly Arg Val Phe Phe Asp Tyr Trp
Gly Gln Gly Thr Leu Val Thr Val Ser Ser -
SEQ. ID. NO. 11
Gln Val Gln Leu Gln Glu Ser Gly Pro Gly Leu Val Lys Pro Ser Glu Thr Leu Ser
Leu Thr Cys Thr Val Ser Gly Tyr Ser Ile Ser Gly Gly Tyr Leu Trp Asn Trp Ile Arg Gln Pro
Pro Gly Lys Gly Leu Glu Trp Ile Gly Tyr Ile Ser Tyr Asp Gly Thr Asn Asn Tyr Lys Pro Ser
Leu Lys Asp Arg Val Thr Ile Ser Val Asp Thr Ser Lys Asn Gln Phe Ser Leu Lys Leu Ser Ser
Val Thr Ala Ala Asp Thr Ala Val Tyr Tyr Cys Ala Arg Tyr Gly Arg Val Phe Phe Asp Tyr Trp
Gly Gln Gly Thr Leu Val Thr Val Ser Ser -
SEQ. ID. NO. 12
Asp Val Leu Met Thr Gln Ile Pro Leu Ser Leu Pro Val Ser Leu Gly Asp Gln Ala
Ser Ile Ser Cys Arg Ser Ser Gln Ser Ile Val His Ser Asn Gly Asn Thr Tyr Leu Gln Trp Tyr
Leu Gln Lys Pro Gly Gln Ser Pro Lys Leu Leu Ile Tyr Lys Val Ser Asn Arg Leu Tyr Gly Val
Pro Asp Arg Phe Ser Gly Ser Gly Ser Gly Thr Asp Phe Thr Leu Lys Ile Ser Ser Val Glu Ala
Glu Asp Leu Gly Val Tyr Tyr Cys Phe Gln Gly Ser His Val Pro Trp Thr Phe Gly Gly Gly Thr
Lys Leu Glu Ile Lys -
SEQ. ID. NO. 13
Asp Val Gln Leu Gln Glu Ser Gly Pro Gly Leu Val Lys Pro Ser Gln Ser Leu Ser
Leu Thr Cys Ser Val Thr Gly Tyr Ser Ile Thr Gly Gly Tyr Leu Trp Asn Trp Ile Arg Gln Phe
Pro Gly Asn Lys Leu Glu Trp Met Gly Tyr Ile Ser Tyr Asp Gly Thr Asn Asn Tyr Lys Pro Ser
Leu Lys Asp Arg Ile Ser Ile Thr Arg Asp Thr Ser Lys Asn Gln Phe Phe Leu Lys Leu Asn Ser
Val Thr Asn Glu Asp Thr Ala Thr Tyr Tyr Cys Ala Arg Tyr Gly Arg Val Phe Phe Asp Tyr Trp
Gly Gln Gly Thr Thr Leu Thr Val Ser Ser -

What is claimed is:

1. A method of treating or preventing a medical condition in a subject comprising: administering a therapeutically effective amount of combination therapeutic comprising a tyrosine kinase inhibitor and an IGF-1R inhibitor or a pharmaceutical composition thereof to said subject, wherein administration of the combination therapeutic results in enhanced therapeutic efficacy relative to administration of the EGFR inhibitor or IGF-1R inhibitor alone, sufficient to treat said patient.

2. The method of claim 1 wherein the tyrosine kinase inhibitor is Erlotinib.

3. The method according to claim 1, wherein said medical condition is Erlotinib resistant cancer.

4. The method of claim 1 wherein the IGF-1R inhibitor or one of its functional fragments is an antibody that specifically binds human IGF-1R and wherein the antibody comprises at least one heavy chain complementary determining region (CDR) of non-human origin and at least one light chain complementary determining region (CDR) derived from a non-human source, wherein said antibody that binds to IGF-IR has at least one of the following properties selected from the group consisting of: a) binding IGF-1R but not IR; (b) binds a hybrid receptor comprising an insulin receptor and insulin growth factor receptor (IR/IGF-1R hybrid-R) but not IR alone; c) inhibiting the binding between a human IGF-1R and IGF-1 and/or IGF-2; (d) binding the hybrid-R and its native ligand, preferably designated herein as IGF1 and/or IGF2 and/or insulin, with an inhibition constant and/or IC50 of less than 100 nM; (e) specifically inhibiting the tyrosine kinase activity of said IGF-1R; (f) specifically inhibiting the tyrosine kinase activity of said hybrid-R; (g) having a binding affinity of 10 nM or less for said hybrid-R; (h) down-regulating IGF-1R expression; (i) down-regulating hybrid-R expression; (j) inhibiting in vivo tumor growth.

5. The method according to claim 4, wherein said IGF-1r antibody comprises a heavy chain and a light chain, wherein the heavy chain comprises at least one CDR comprising an amino acid sequence selected from the group consisting of SEQ ID NOs. 4, 5 or 6 and the light chain comprises at least one CDR comprising an amino acid sequence selected from the group consisting of SEQ ID NOs. 1, 2 or 3.

6. The method of claim 1 wherein the anti-IGF-1R antibody is selected from the group consisting of dalotuzumab, figitumumab, cixutumumab, SHC 717454, Roche R1507 and Amgen AMG479.

7. The method according to claim 3, wherein said humanized antibody, or one of its functional fragments, comprises a light chain comprising the amino acid sequence selected from the group consisting of SEQ ID No. 7 or 8, or a heavy chain comprising the amino acid sequence selected from the group consisting of SEQ ID NOs.: 9, 10 or 11.

8. The method of claim 7, wherein the anti-IGF-1R antibody is dalotuzumab.

9. The method according to claim 1 wherein the tyrosine kinase inhibitor is Erlotinib and is administered in a dose between 10 mg and 400 mg.

10. The method according to claim 1 wherein the tyrosine kinase inhibitor is administered in a dose between 100-300 mg/kg weekly.

11. The method according to claim 1, wherein the combination therapeutic comprising said tyrosine kinase inhibitor and said IGF-1R inhibitor is dosed as follows: tyrosine kinase is dosed at about 150 mg/kg and the IGF-1R inhibitor is administered at a dose of 10 mg/kg weekly.

12. The method according to 11, wherein the tyrosine kinase inhibitor is Erlotinib and is administered five times a week.

13. The method of claim 11 wherein the dalotuzumab is administered intravenously at a dose of 10 mg/kg.

14. The method of claim 11 wherein the dalotuzumab is administered once a week.

15. The method of claim 11 wherein the dalotuzumab is administered once every other week.

16. The method of claim 1 wherein the IGF1R inhibitor is administered in association with one or more further chemotherapeutic agents or a pharmaceutical composition thereof.

17. The method of claim 16 wherein the further chemotherapeutic agent is one or more members selected from the group consisting of teniposide 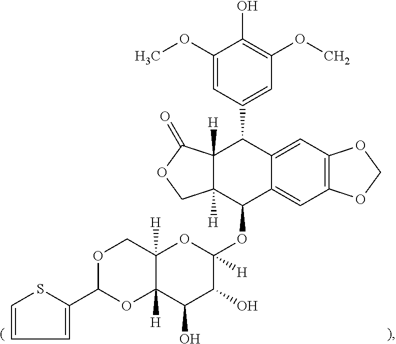
cisplatin 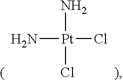
carboplatin 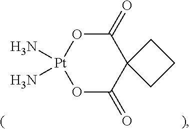
etoposide 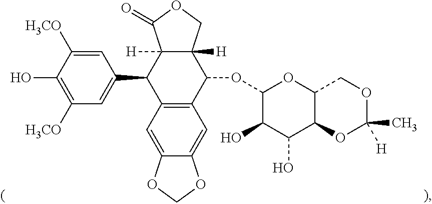
doxorubicin 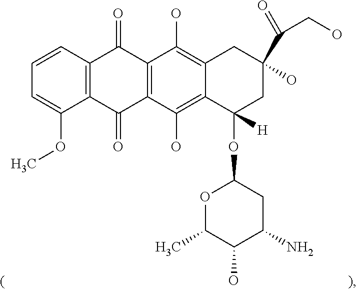
any liposomal formulation thereof, cyclophosphamide 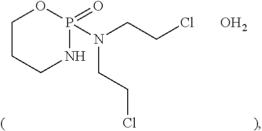
13-cis-retinoic acid 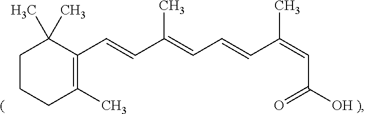
ifosfamide 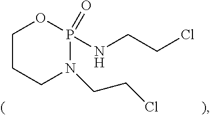
gemcitabine 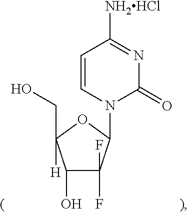
irinotecan 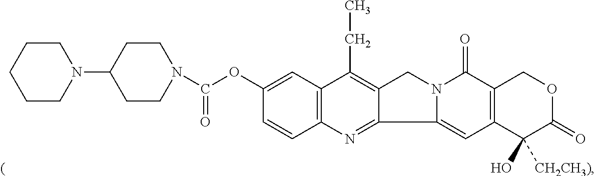 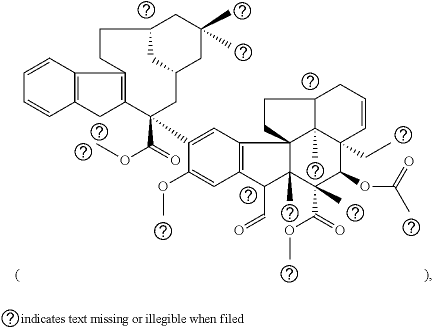
vincristine ( ), dactinomycin 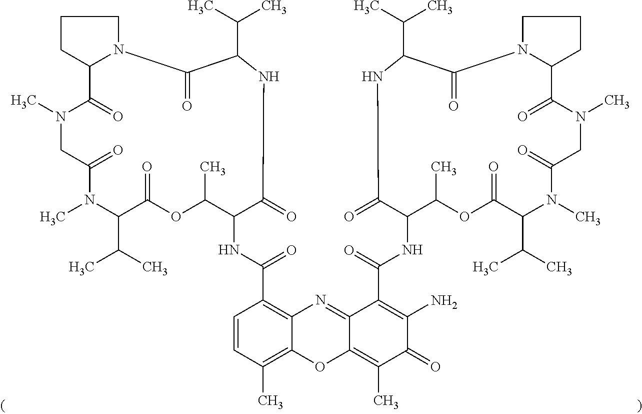
calcitriol, and methotrexate

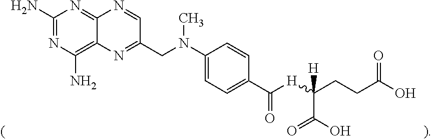

18. The method of claim 17 wherein the IGF1R inhibitor and the further anti-cancer therapeutic agent are administered simultaneously.

19. The method of claim 17 wherein the IGF1R inhibitor and the further anti-cancer therapeutic agent are administered non-simultaneously.

20. The method of claim 17 wherein the IGF1R inhibitor is administered in association with an anti-cancer therapeutic procedure.

21. The method of claim 17 wherein the anti-cancer therapeutic procedure is surgical tumorectomy and/or anti-cancer radiation treatment.

22. The method of claim 1 wherein the IGF1R inhibitor is selected from the group consisting of 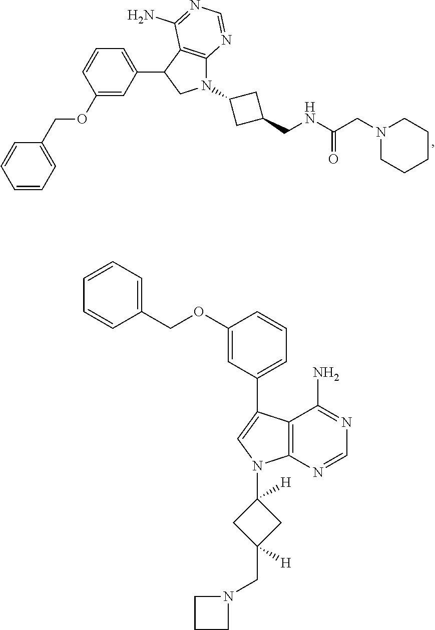
and an isolated antibody that binds specifically to human IGF1R or an antigen-binding fragment thereof.
